(19) United States
(12) Patent Application Publication
(10) Pub. No.: US 2005/0075795 A1
(43) Pub. Date: Apr. 7, 2005

(54) Crystal structure of 3', 5'-cyclic nucleotide phosphodiesterase (PDE1B) and uses thereof

(75) Inventor: Jayvardhan Pandit, Mystic, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/815,390

(22) Filed: Mar. 31, 2004

Related U.S. Application Data
(60) Provisional application No. 60/458,946, filed on Mar. 31, 2003.

(51) Int. Cl. G06F 19/00; G01N 33/48; G01N 33/50; G01N 31/00

(52) U.S. Cl. 702/20

(57) ABSTRACT

Crystal structures of phosphodiesterase 1B (PDE1B), and the 3-D atomic coordinates of the PDE1B binding domain, are described and used to obtain PDE1B ligands, including PDE1B inhibitors. The inhibitors are formulated into pharmaceutical compositions and used to treat various psychological disorders.

This application claims the benefit of U.S. Provisional Application No. 60/458,946, filed on Mar. 31, 2003 and incorporated herein by reference in its entirety.

### FIELD OF THE INVENTION

The present invention relates to crystalline compositions of mammalian 3′, 5′-Cyclic Nucleotide Phosphodiesterase (PDE1B), methods of preparing said compositions, methods of determining the 3-D X-ray atomic coordinates of said composition, methods of identifying ligands of PDE1B using structure based drug design, the use of the 3-D crystal structure to design, modify and assess the activity of potential inhibitors, and to the use of such inhibitors for example, as psychotherapeutics.

### BACKGROUND OF THE INVENTION

Cyclic nucleotide second messengers (cAMP and cGMP) play a central role in signal transduction and regulation of physiologic responses. Their intracellular levels are controlled by the complex superfamily of cyclic nucleotide phosphodiesterase (PDE) enzymes. The PDE superfamily is comprised of metallophosphohydrolases (e.g., Mg²⁺, and Zn²⁺) that specifically cleave the 3′,5′-cyclic phosphate moiety of cAMP and/or cGMP to produce the corresponding 5′-nucleotide. The sensitivity of physiological processes to cAMP/cGMP signals requires that their levels be precisely maintained within a relatively narrow range in order to provide for optimal responsiveness in a cell. Cyclic nucleotide PDEs provide the major pathway for eliminating the cyclic nucleotide signal for the cell. PDEs are critical determinants for modulation of cellular levels of cAMP and/or cGMP by many stimuli.

Members of the PDE superfamily differ substantially in their tissue distributions, physicochemical properties, substrate and inhibitor specificities and regulatory mechanisms. Based on differences in primary structure of known PDEs, they have been subdivided into two major classes, class I and class II. To date, no mammalian PDE has been included in class II. Class I contains the largest number of PDEs and includes all known mammalian PDEs. Each class I PDE contains a conserved segment of ˜250-350 amino acids in the carboxyl-terminal portion of the proteins, and this segment has been demonstrated to include the catalytic site of these enzymes. All known class I PDEs are contained within cells and vary in subcellular distribution, with some being primarily associated with the particulate fraction of the cytoplasmic fraction of the cell, others being evenly distributed in both compartments.

PDEs from mammalian tissues have been subdivided into 11 families that are derived from separate gene families. The families are named PDE1, PDE2, PDE3, . . . to PDE 11. Within each family, there may be isoenzymes such as PDE1A, PDE1B and PDE1C, and PDE10A1 and PDE10A2. PDEs within a given family may differ significantly but the members of each family are functionally related to each other through similarities in amino acid sequences, specificities and affinities for cGMP (PDE5, PDE6, and PDE9) and cAMP (PDE4, PDE7, and PDE8) or accommodation of both (PDE1, PDE2, PDE3, PDE10, and PDE11), inhibitor specificities, and regulatory mechanisms.

Comparison of the amino acid sequences of PDEs suggests that all PDEs may be chimeric multidomain proteins possessing distinct domains that provide for catalysis and a number of regulatory functions. The amino acid sequences of all mammalian PDEs identified to date include a highly conserved region of approximately 270 amino acids located in the carboxy terminal half of the proteins. (Charbonneau, et al., Proc. Natl. Acad., Sci. (USA) 83:9308-9312 (1986)). The conserved domain includes the catalytic site for cAMP and/or cGMP hydrolysis and two putative metal (presumably zinc) binding sites as well as family specific determinants.(Beavo, Physiol. Rev. 75: 725-748 (1995); Francis, et al., J. Biol. Chem. 269:22477-22480 (1994)). The amino terminal region of the various PDEs are highly variable and include other family specific determinants such as : (i) calmodulin binding sites (PDE1); (ii) non-catalytic cGMP binding sites (PDE2, PDE5, PDE6); (iii) membrane targeting sites (PDE4); (iv) hydrophobic membrane association sites (PDE3); and (v) phosphorylation sites for either the calmodulin-dependent kinase (II) (PDE1), the cAMP-dependent kinase (PDE1, PDE3, PDE4), or the cGMP dependent kinase (PDE5) (Beavo, Physiol. Rev. 75:725-748 (1995); Manganiello, et al., Arch. Biochem. Acta 322: 1-13 (1995); Conti, et al., Physiol. Rev. 75:723-748 (1995); WO 99/42596).

It has been demonstrated that human PDE1B1 mRNA is expressed in a variety of tissue types. PDE1B is most readily detected in human brain. In situ hybridization and immunocytochemistry demonstrated high levels of PDE1B mRNA and protein in the caudate putamen, nucleus accumbens, and olfactory tubercle. The level of mRNA in these regions is 4 to 30-fold more than other brain regions (Polli and Kincaid, PNAS, 89: 11079-83, 1992). Ubiquitous expression of PDE1B was observed within the caudate putamen. Immunological and biochemical data suggest that PDE1B accounts for 30-40% of total CaM-PDE in whole mouse brain (Polli and Kincaid, J. Neurosci., 14: 1251-61, 1994). It is also readily detected in human heart. (Yu et al., “Identification and characterisation of a human calmodulin-stimulated phosphodiesterase PDE1B1.” Cell. Signal. 9:519-529(1997)). This expression pattern is strikingly similar to D1 dopamine receptor and correlates strongly with brain areas that are richest in dopaminergic innervation, suggesting an important role in antagonism of cAMP-regulated signaling in dopaminoceptive neurons. PDE1B inhibitors therefore, should enhance dopaminergic signaling. Augmentation of D1 function in the striatal areas may provide benefit in a variety of cardiac disorders, and CNS disorders including schizophrenia, depression, bipolar illness, dementia, psychostimulant withdrawal, as well as Parkinson's Disease.

### SUMMARY OF THE INVENTION

The present invention relates generally to crystalline compositions of PDE1B, methods of preparing said compositions, methods of determining the 3-D X-ray atomic coordinates of said crystalline compositions, methods of using said atomic coordinates in conjunction with computational methods to identify binding site(s), elucidating the 3-D structure of homologues or variants of PDE1B, or identifying ligands which interact with said binding site(s) to agonize or antagonize the biological activity of PDE1B, methods for identifying inhibitors of PDE1B, pharmaceutical compositions of inhibitors so identified, and methods of treatment of psychotherapeutic disorders using said pharmaceutical compositions.

In a preferred embodiment the invention provides crystalline compositions of the catalytic region of PDE1B.

In certain embodiments, the method further comprises refining and evaluating said full or partial 3-D coordinates. This method may thus be used to generate 3-dimensional structures for proteins for which heretofore 3-dimensional atomic coordinates have not been determined. Depending on the extent of sequence homology, the newly generated structure may help to elucidate enzymatic mechanisms, or be used in conjunction with other molecular modeling techniques in structure based drug design.

In another aspect, the present invention provides a method for identifying inhibitors, ligands, and the like of PDE1B by providing the coordinates of a molecule of PDE1B to a computerized modeling system; identifying chemical entities that are likely to bind to or interfere with the molecule (e.g., by screening a small molecule library); and, optionally, procuring or synthesizing and assaying the compounds or analogues derived therefrom for bioactivity.

In certain embodiments, the information obtained by this method is used to iteratively refine or modify the structure of the original ligand. Thus, once a ligand is found to modulate the activity of said enzyme, the structural aspects of the ligand may be modified to generate a structural analog of the ligand. This analog can then be used in the above method to identify better binding ligands. One of ordinary skill in the art will know the various ways a structure may be modified.

In certain embodiments, the ligand is a selective inhibitor of PDE1B.

Thus, in a first aspect, the present invention relates to phosphodiesterase 1B (PDE1B) crystals.

In a second aspect, the present invention relates to crystals of a PDE1B/PDE1B ligand complex.

In a third aspect, the present invention relates to polypeptides comprising the amino acid sequence set forth in SEQ ID NO: 1 or a homologue or variant thereof, wherein the molecules are arranged in a crystalline manner belonging to space group P4₃2₁2 with unit cell dimensions a=87.47 Å, b=87.47 Å, c=135.03 Å, α=β=γ=90.0°, and which effectively diffracts X-rays for determination of the atomic coordinates of PDE1B polypeptide to a resolution of about 1.8 Å.

In a fourth aspect, the present invention relates to polypeptide consisting essentially of the catalytic domain of PDE1B.

In a fifth aspect, the present invention relates to computers for producing a three-dimensional representation of a polypeptide with an amino acid sequence spanning amino acids Thr142 to Gln507 listed in SEQ ID NO: 1, or a homologue, or a variant thereof comprising a computer-readable data storage medium comprising a data storage material encoded with computer-readable data, wherein said data comprises the structure coordinates of FIG. 4, or portions thereof, a working memory for storing instructions for processing said computer-readable data, a central-processing unit coupled to said working memory and to said computer-readable data storage medium for processing said computer-machine readable data into said three-dimensional representation, and a display coupled to said central-processing unit for displaying said representation.

In a sixth aspect, the present invention relates to computers for producing a three-dimensional representation of a molecule or molecular complex comprising the atomic coordinates in FIG. 4 comprising a computer-readable data storage medium comprising a data storage material encoded with computer-readable data, wherein said data comprises the structure coordinates of FIG. 4, or portions thereof, a working memory for storing instructions for processing said computer-readable data, a central-processing unit coupled to said working memory and to said computer-readable data storage medium for processing said computer-machine readable data into said three-dimensional representation, and a display coupled to said central-processing unit for displaying said representation.

In a seventh aspect, the present invention relates to computers for producing a three-dimensional representation of a molecule or molecular complex comprising the atomic coordinates having a root mean square deviation of less than 2.0, 1.7, 1.5, 1.2, 1.0, 0.7, 0.5, or 0.2 Å from the atomic coordinates for the carbon backbone atoms listed in FIG. 4 comprising a computer-readable data storage medium comprising a data storage material encoded with computer-readable data, wherein said data comprises the structure coordinates of FIG. 4, or portions thereof, a working memory for storing instructions for processing said computer-readable data, a central-processing unit coupled to said working memory and to said computer-readable data storage medium for processing said computer-machine readable data into said three-dimensional representation, and a display coupled to said central-processing unit for displaying said representation.

In a eighth aspect, the present invention relates to computers for producing a three-dimensional representation of a molecule or molecular complex comprising a binding site defined by the structure coordinates in FIG. 4, or a the structural coordinates of a portion of the residues in FIG. 4, or the structural coordinates of one or more PDE1B amino acids in SEQ ID NO: 1 selected from His223, His373, Thr385, Leu388, Ser420, Gln421, and Phe424, wherein said computer comprises a computer-readable data storage medium comprising a data storage material encoded with computer-readable data, wherein said data comprises the structure coordinates of FIG. 4, or portions thereof, a working memory for storing instructions for processing said computer-readable data, a central-processing unit coupled to said working memory and to said computer-readable data storage medium for processing said computer-machine readable data into said three-dimensional representation, and a display coupled to said central-processing unit for displaying said representation.

In a ninth aspect, the present invention relates to methods for generating the 3-D atomic coordinates of protein homologues of PDE1B using the X-ray coordinates of PDE1B described in FIG. 4, said methods comprising identifying the sequences of one or more proteins which are homologues of PDE1B, aligning the homologue sequences with the sequence of PDE1B (SEQ ID NO:1), identifying structurally conserved and structurally variable regions between the homologue sequences, and PDE1B (SEQ ID NO:1), generating 3-D coordinates for structurally conserved residues, variable regions and side-chains of the homologue sequences from those of PDE1B, and combining the 3-D coordinates of the conserved residues, variable regions and side-chain conformations to generate a full or partial 3-D coordinates for said homologue sequences.

In a tenth aspect, the present invention relates to methods for identifying a potential ligands for PDE1B, or homologues, analogues or variants thereof, comprising the steps of displaying three dimensional structure of PDE1B enzyme, or portions thereof, as defined by atomic coordinates in FIG. 4, on a computer display screen, optionally replacing one or more PDE1B enzyme amino acid residues listed in SEQ ID NO:1, or one or more of the amino acids listed in Tables 1-3, or one or more amino acid residues selected from His223, His373, Thr385, Leu388, Ser420, Gln421, and Phe424, in said three-dimensional structure with a different naturally occurring amino acid or an unnatural amino acid, employing said three-dimensional structure to design or select said ligand, contacting said ligand with PDE1B, or variant thereof, in the presence of one or more substrates, and measuring the ability of said ligand to modulate the activity PDE1B.

In a eleventh aspect, the present invention relates to methods for treating psychological disorders comprising administering to a patient in need of treatment the pharmaceutical compositions of ligands identified by structure-based drug design using the atomic coordinates substantially similar to, or portions of, the coordinated listed in FIG. 4.

In a twelfth aspect, the present invention relates to expression vectors useful in a method for preparing a purified catalytic domain of PDE1B comprising a polypeptide with an amino acid sequence spanning amino acids Thr142 to Gln507 listed in SEQ ID NO:1, or a homologue or variant thereof.

### BRIEF DESCRIPTION OF THE DRAWINGS

FIG. 1 is an orthogonal view of the structure of PDE1B in ribbon representation. Compound 109 is shown in ball-and-stick representation, and bound Zn and Mg ions are shown as balls. N- and C- termini of the polypeptide are labelled.

FIG. 2 is another orthogonal view of the structure of PDE1B.

FIG. 3 is a schematic diagram showing the interactions of Compound 109 with PDE1B.

FIG. 4 is a list of the X-ray coordinates of the PDELB C-terminal catalytic domain crystal as described in the Examples.

### DETAILED DESCRIPTION OF THE INVENTION

The present invention relates to crystalline compositions of PDE1B, methods of preparing said compositions, methods of determining the 3-D X-ray atomic coordinates of said crystalline compositions, and methods of using said atomic coordinates in conjunction with computational methods to identify binding site(s), or identify ligands which interact with said binding site(s) to agonize or antagonize PDE1B.

I. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The term “affinity” as used herein refers to the tendency of a molecule to associate with another. The affinity of a drug is its ability to bind to its biological target (receptor, enzyme, transport system, etc.) For pharmacological receptors, affinity can be thought of as the frequency with which the drug, when brought into the proximity of a receptor by diffusion, will reside at a position of minimum free energy within the force field of that receptor.

The term “agonist” as used herein refers to an endogenous substance or a drug that can interact with a receptor and initiate a physiological or a pharmacological response characteristic of that receptor (contraction, relaxation, secretion, enzyme activation, etc.)

The term “analog” as used herein refers to a drug or chemical compound whose structure is related in some way to that of another drug or chemical compound, but whose chemical and biological properties may be quite different.

The term “antagonist” as used herein refers to a drug or a compound that opposes the physiological effects of another. At the receptor level, it is a chemical entity that opposes the receptor- associated responses normally induced by another bioactive agent.

As used herein the term “binding site” refers to a specific region (or atom) in a molecular entity that is capable of entering into a stabilizing interaction with another molecular entity. In certain embodiments the term also refers to the reactive parts of a macromolecule that directly participate in its specific combination with another molecule. In certain other embodiments, a binding site may be comprised or defined by the three dimensional arrangement of one or more amino acid residues within a folded polypeptide. In certain embodiments, the binding site further comprise prosthetic groups, water molecules or metal ions which may interact with one or more amino acid residues. Prosthetic groups, water molecules, or metal ions may be apparent from X-ray crystallographic data, or may be added to an apoprotein or enzyme using in silico methods.

The term “catalytic domain” as used herein, refers to the catalytic domain of the PDE1 class of enzymes, which feature a conserved segment of 250-350 amino acids in the carboxy-terminal portion of the proteins, wherein this segment has been demonstrated to include the catalytic site of these enzymes. This conserved catalytic domain extends approximately from residue 150 to residue 510 of the full-length enzyme.

“To clone” as used herein, as will be apparent to skilled artisan, may be meant as obtaining exact copies of a given polynucleotide molecule using recombinant DNA technology. Furthermore, “to clone into” may be meant as inserting a given first polynucleotide sequence into a second polynucleotide sequence, preferably such that a functional unit combining the functions of the first and the second polynucleotides results, for example, without limitation, a polynucleotide from which a fusion protein may be translationally provided, which fusion protein comprises amino acid sequences encoded by the first and the second polynucleotide sequences. Details of molecular cloning can be found in a number of commonly used laboratory protocol books such as Molecular Cloning: A Laboratory Manual, 2nd Ed., ed. by Sambrook, Fritsch and Maniatis (Cold Spring Harbor Laboratory Press: 1989).

The term “co-crystallization” as used herein is taken to mean crystallization of a preformed protein/ligand complex.

The term “complex” or “co-complex” are used interchangeably and refer to a PDELB molecule, or a variant, or homologue of PDE1B in covalent or non-covalent association with a substrate, or ligand.

The term “contacting” as used herein applies to in silico, in vitro, or in vivo experiments.

As used herein, the terms “gene”, “recombinant gene” and “gene construct” refer to a nucleic acid comprising an open reading frame encoding a polypeptide, including both exon and (optionally) intron sequences. The term “intron” refers to a DNA sequence present in a given gene which is not translated into protein and is generally found between exons.

The term “high affinity” as used herein means strong binding affinity between molecules with a dissociation constant K_{D} of no greater than 1 μM. In a preferred case, the K_{D} is less than 100 nM, 10 nM, 1 nM, 100 pM, or even 10 pM or less. In a most preferred embodiment, the two molecules can be covalently linked (K_{D} is essentially 0).

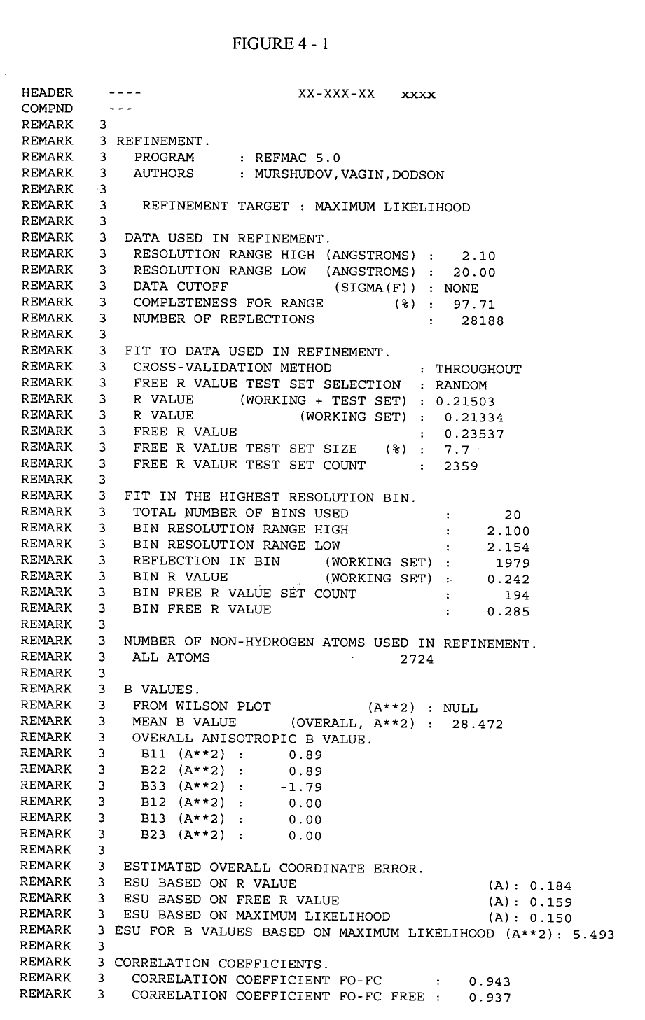

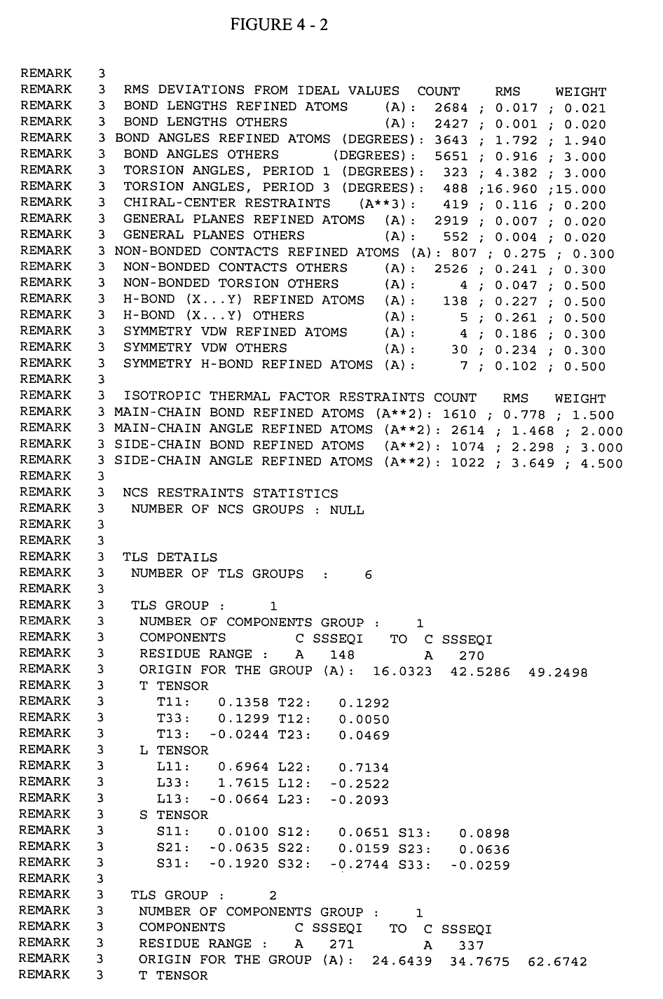

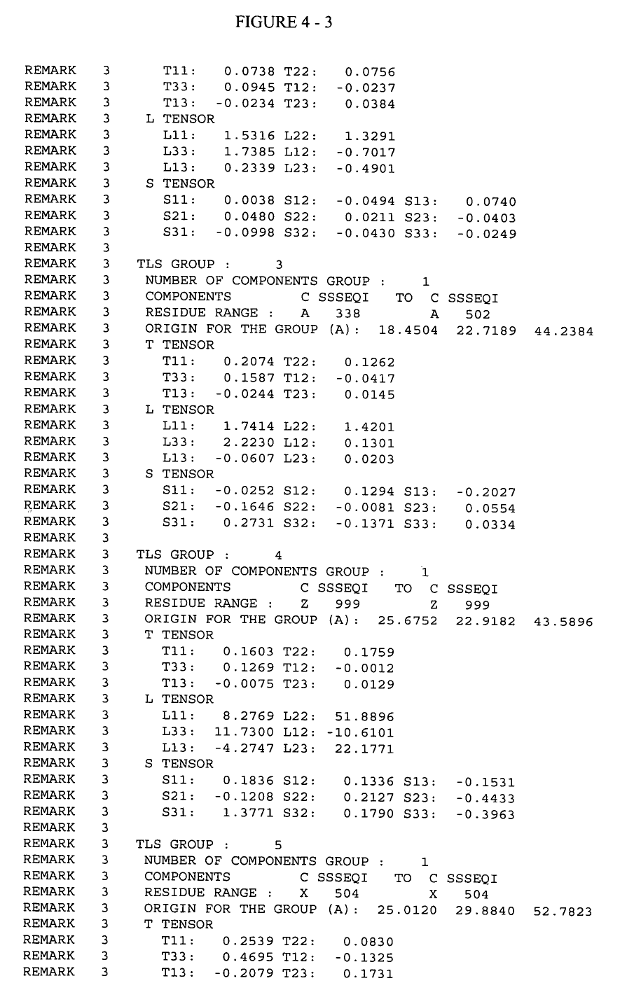

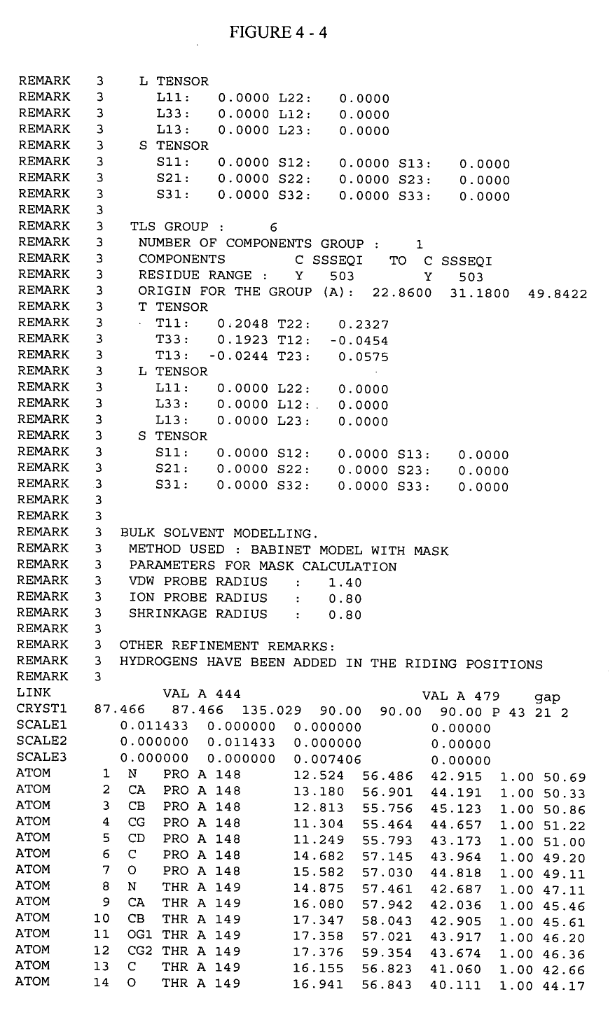

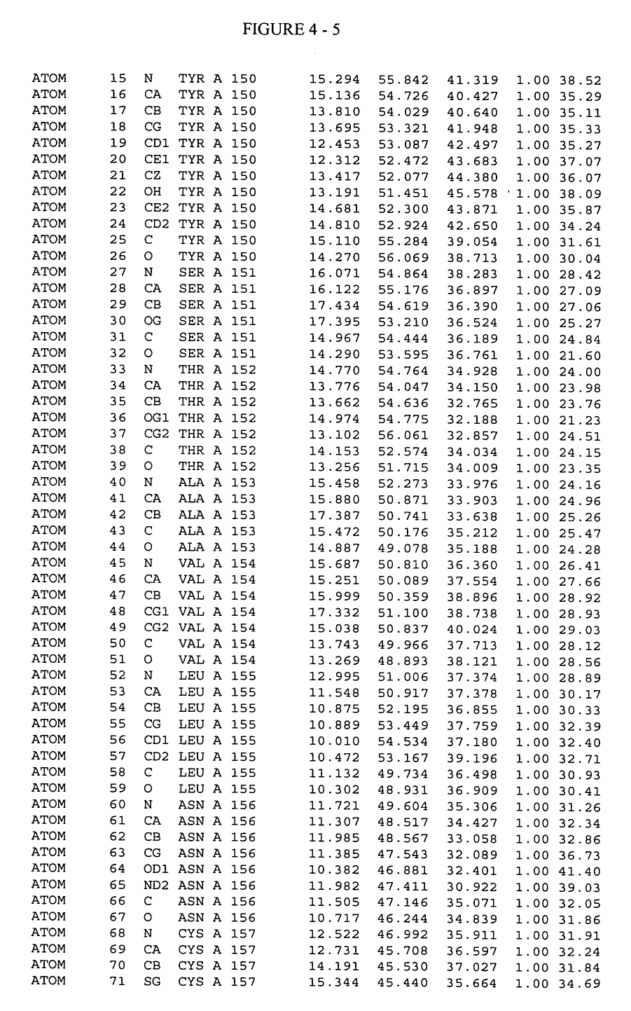

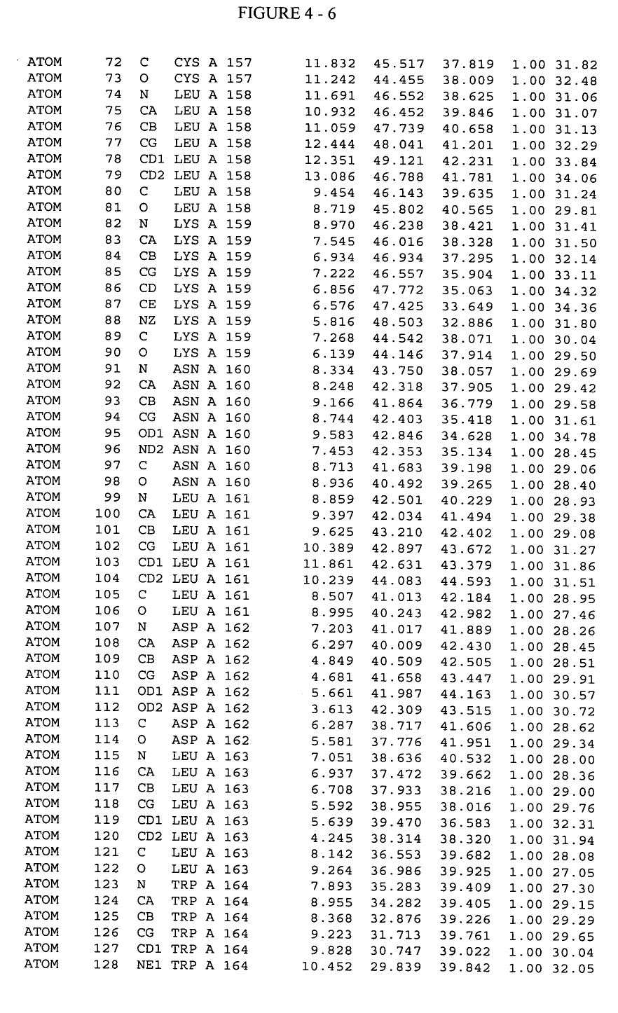

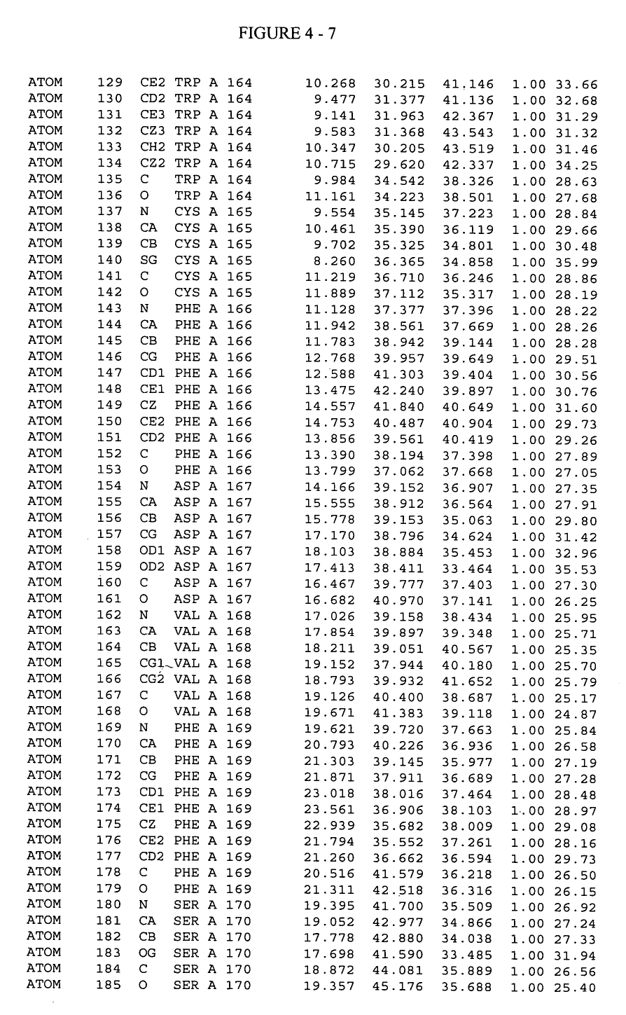

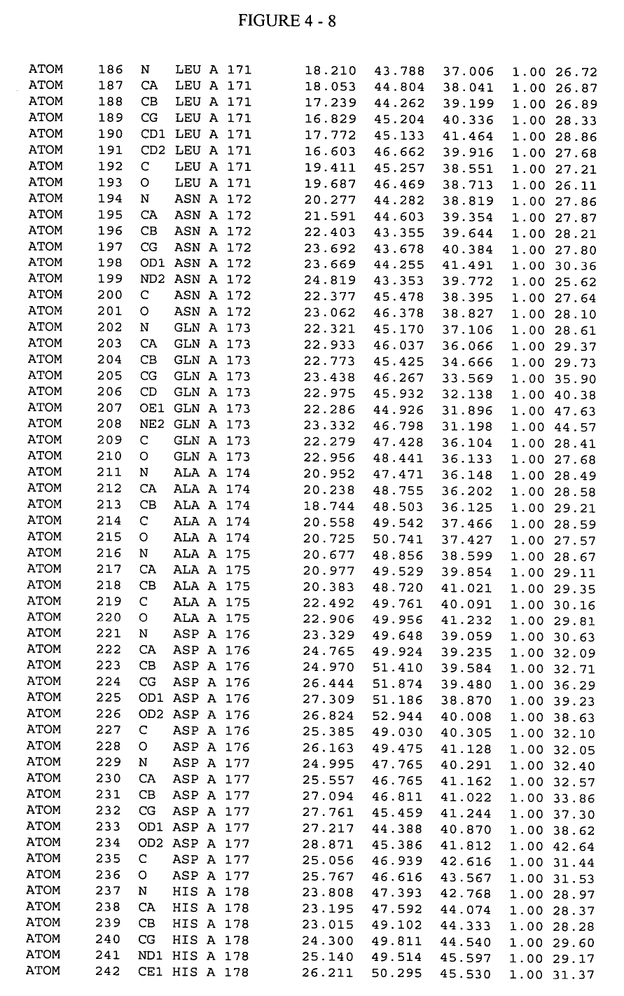

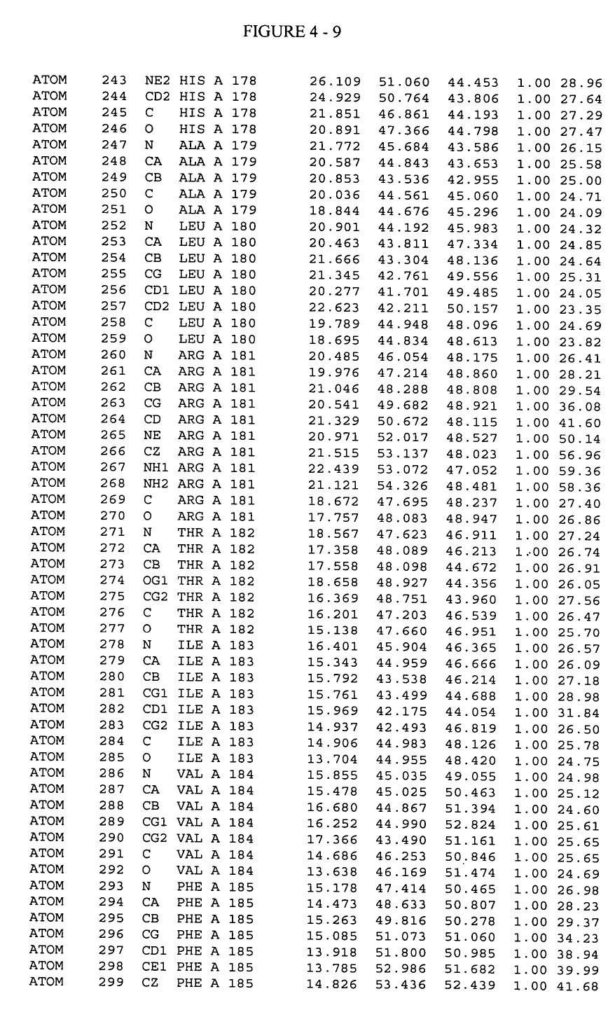

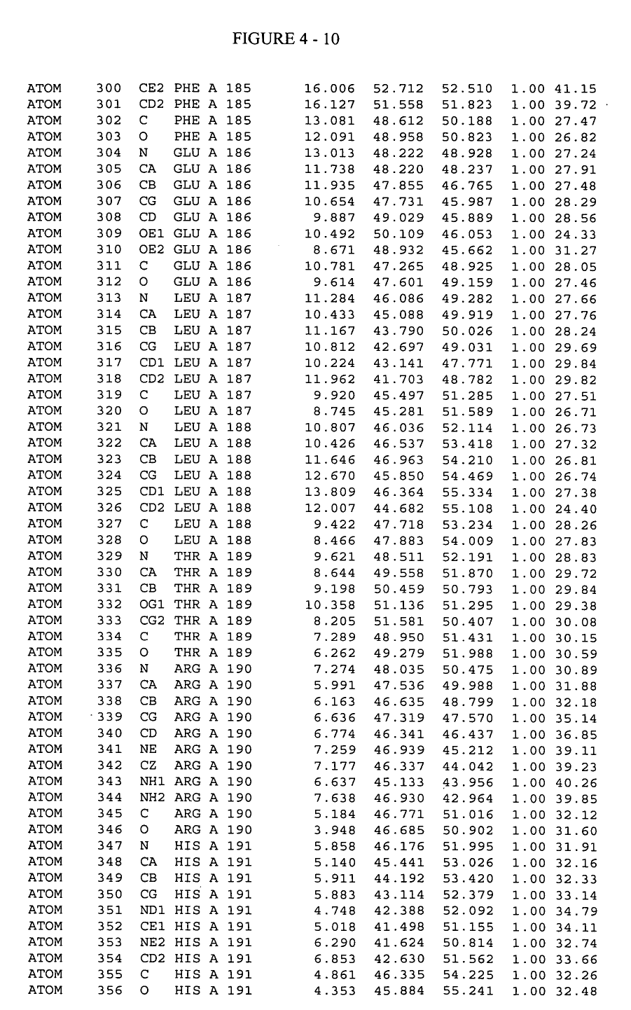

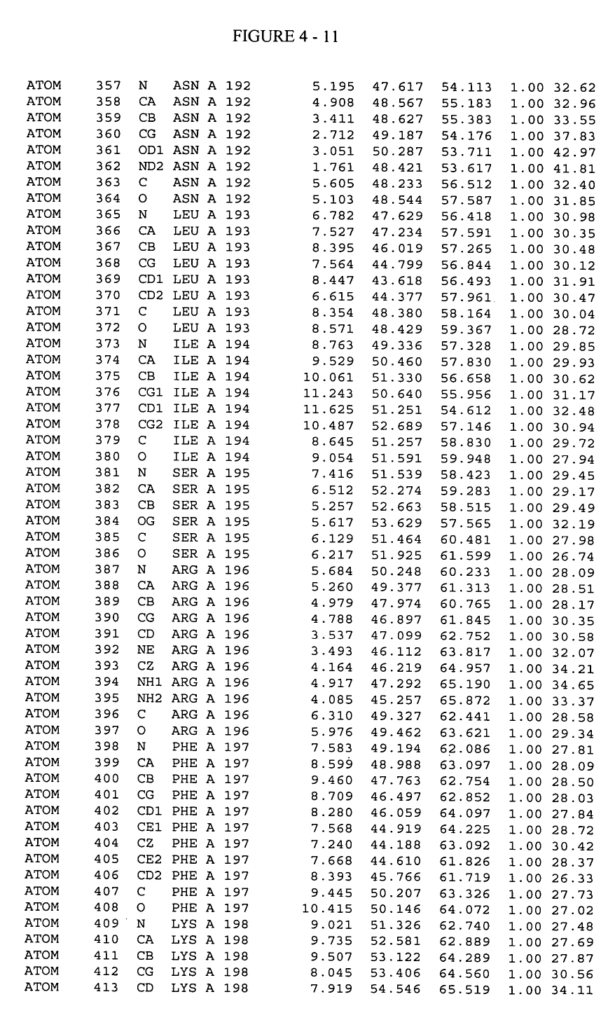

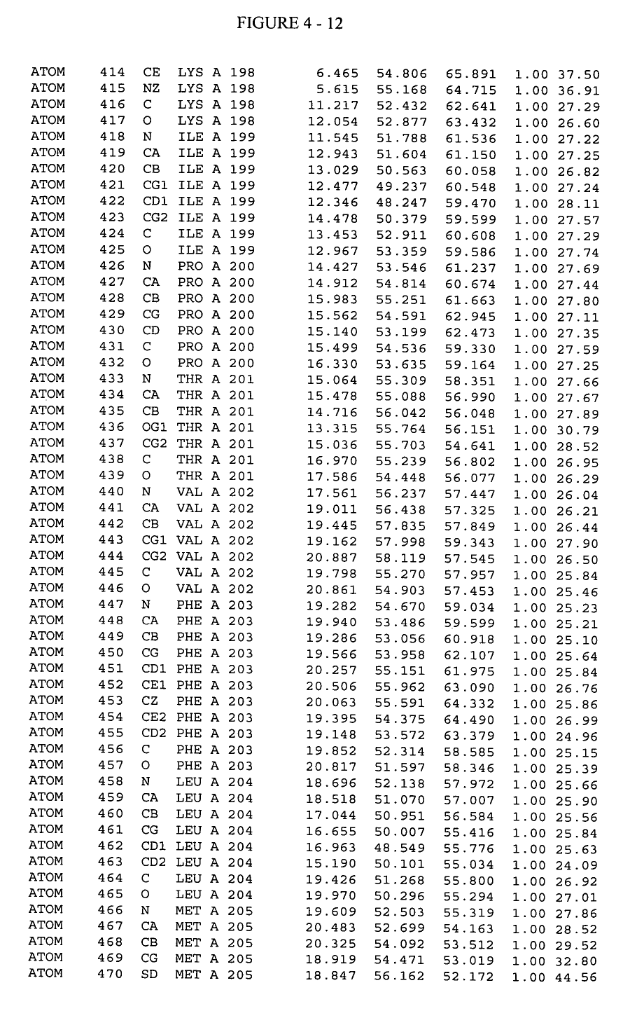

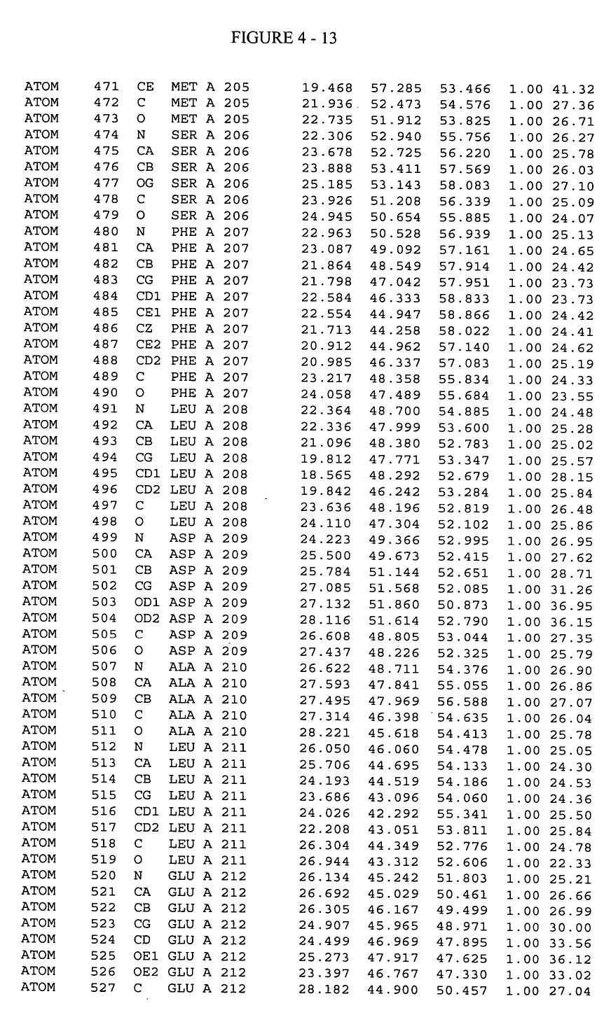

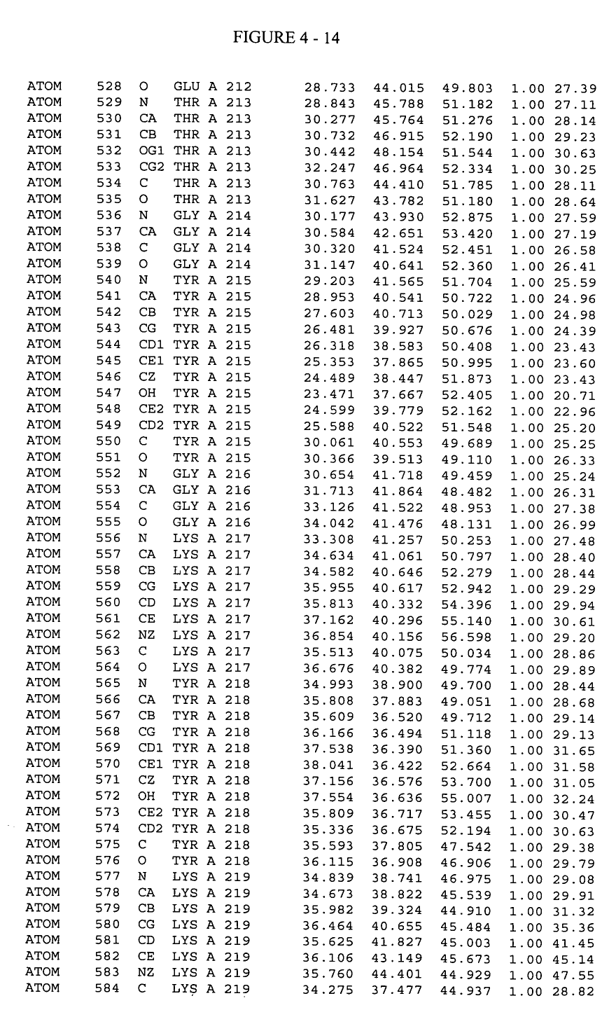

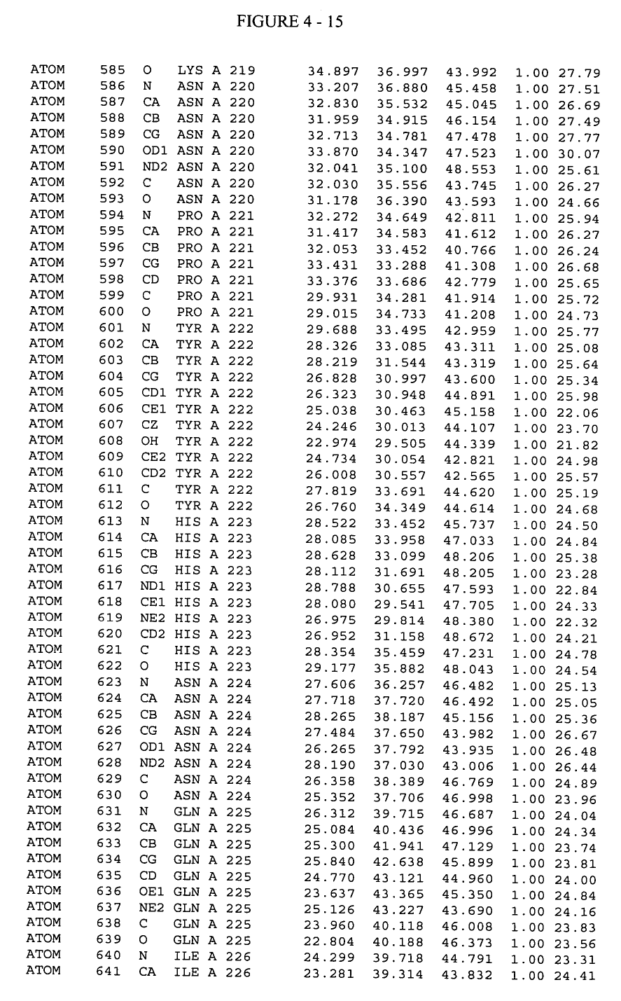

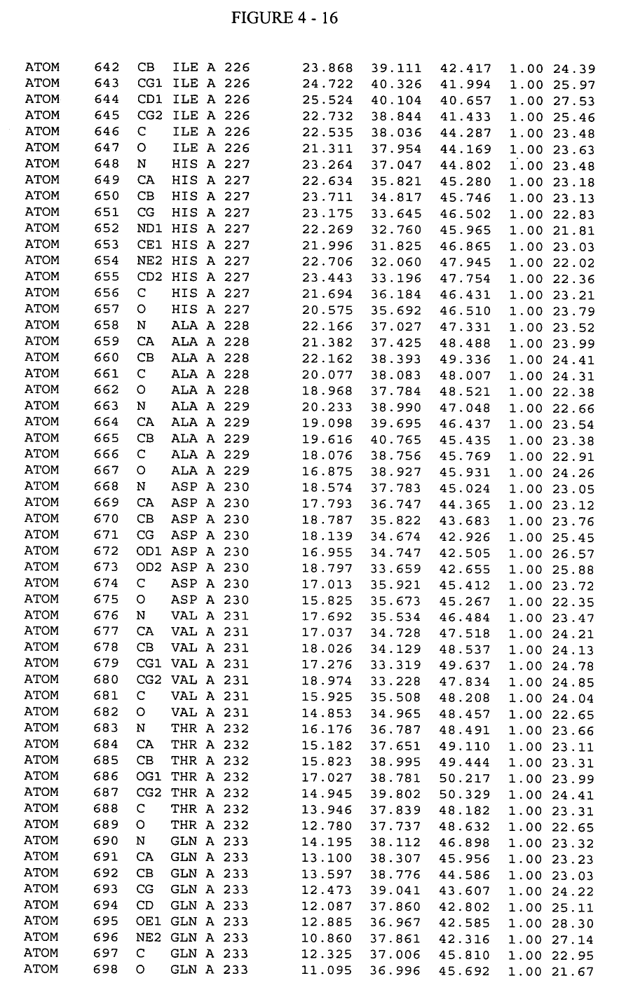

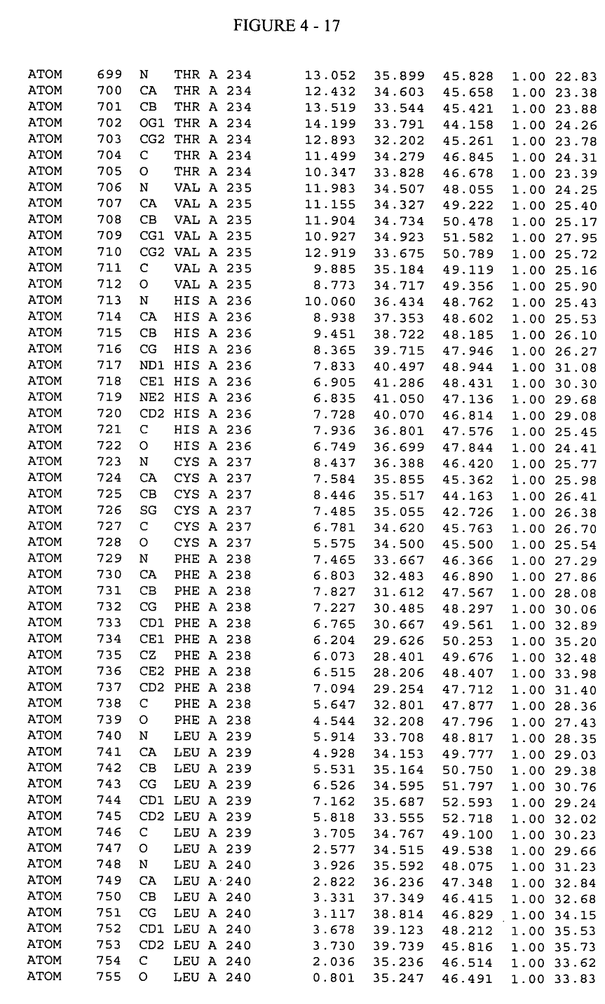

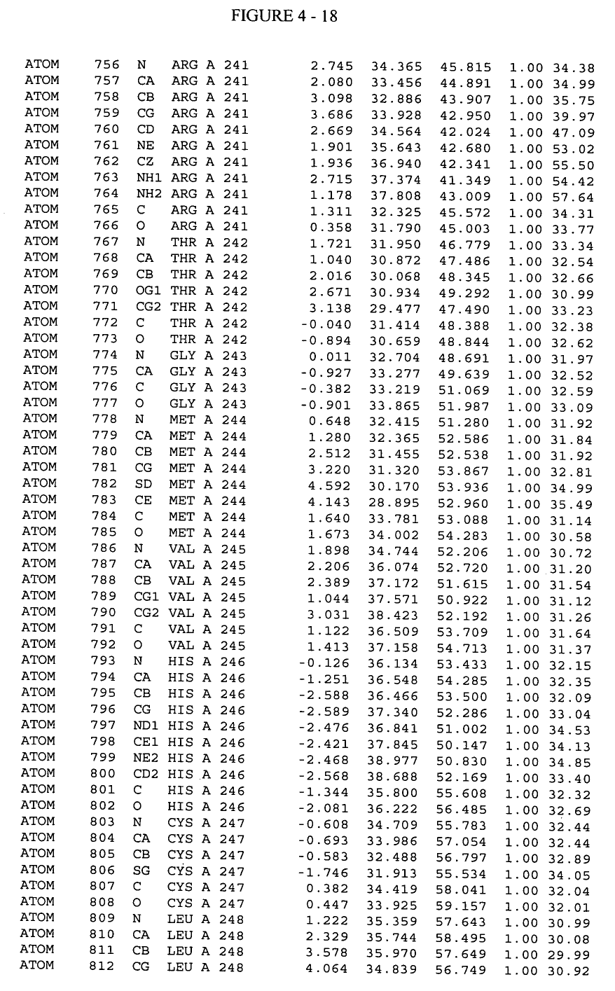

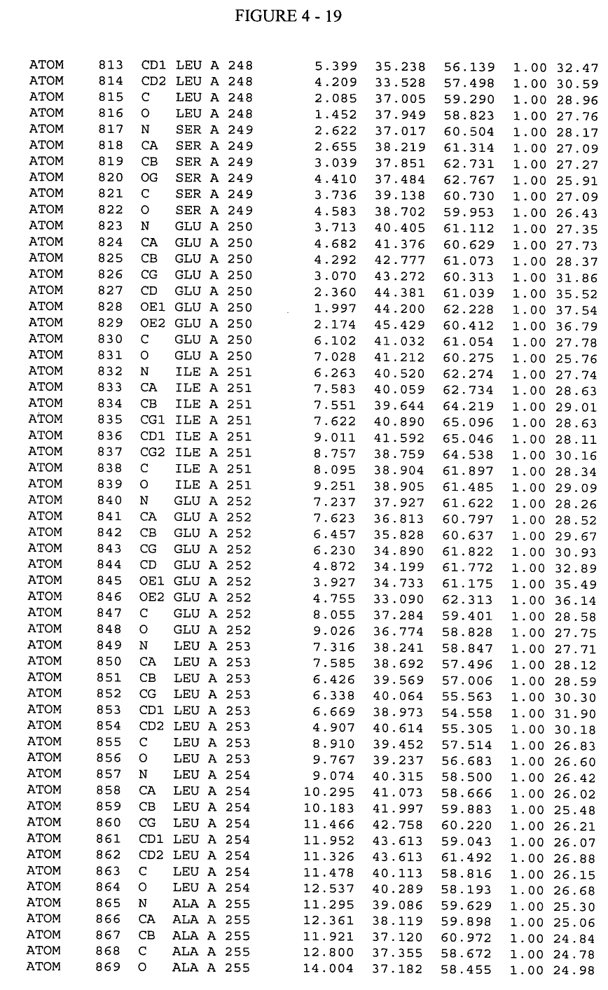

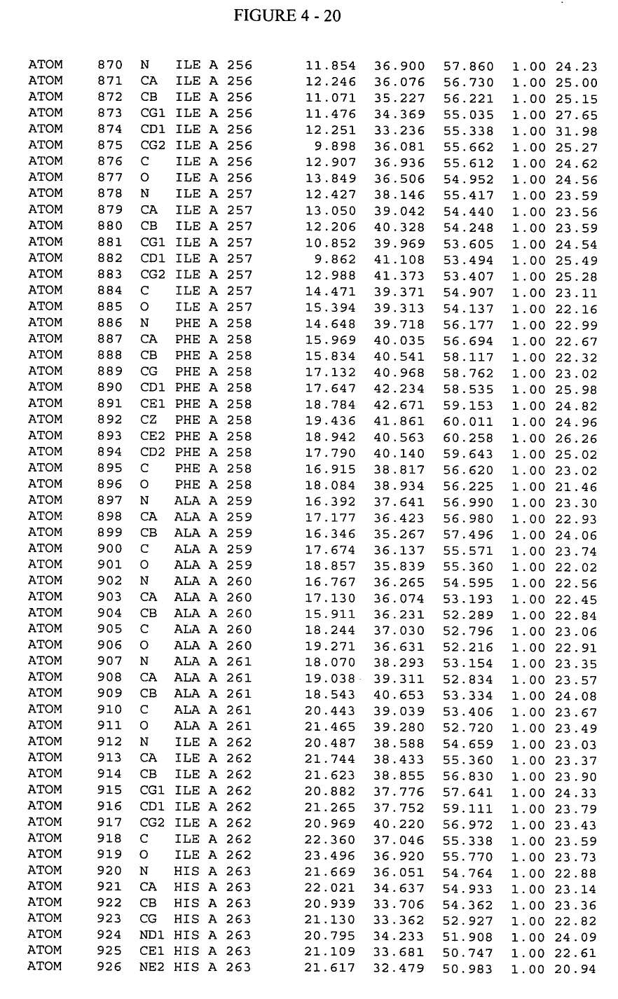

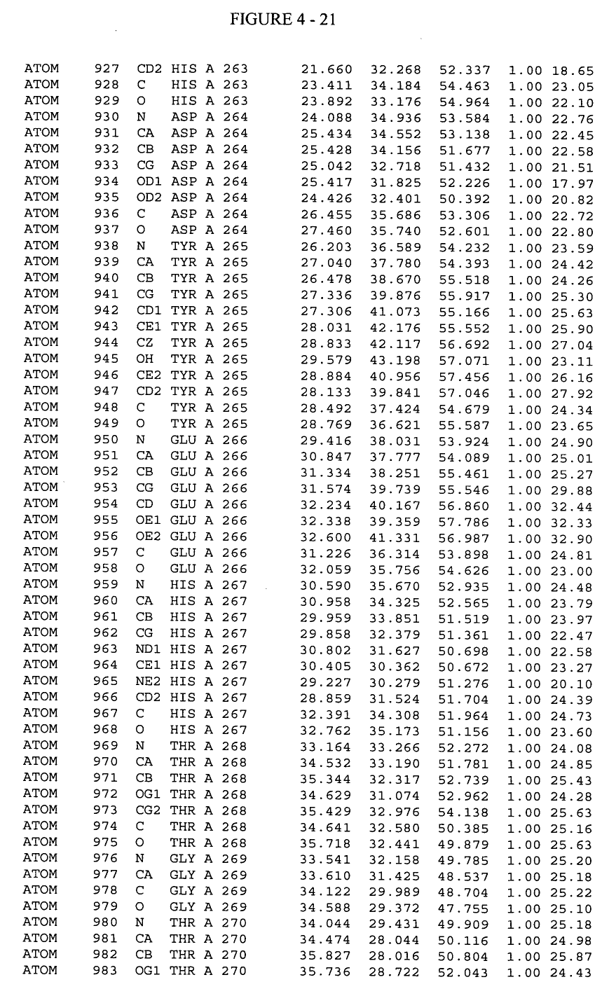

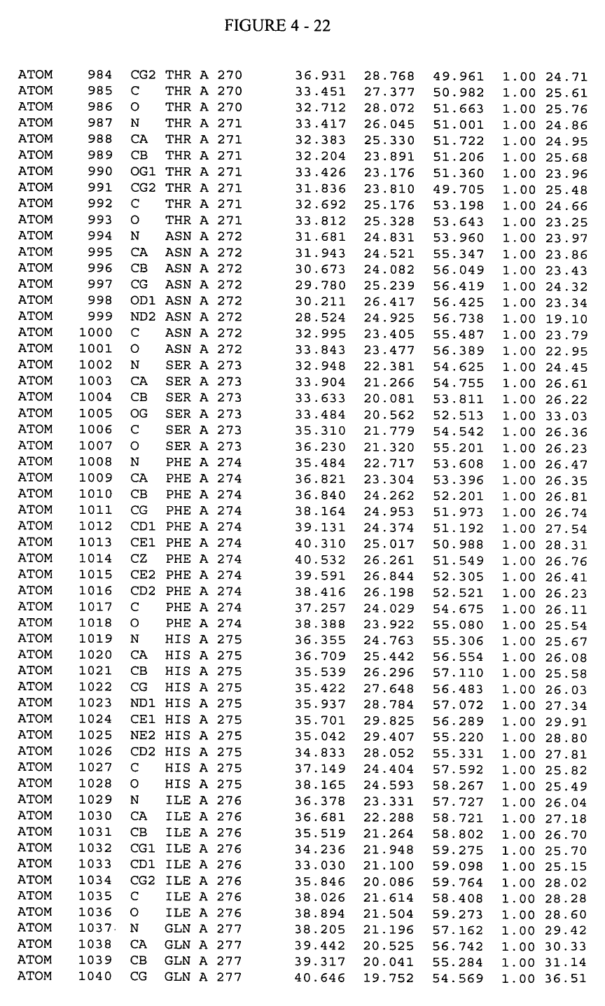

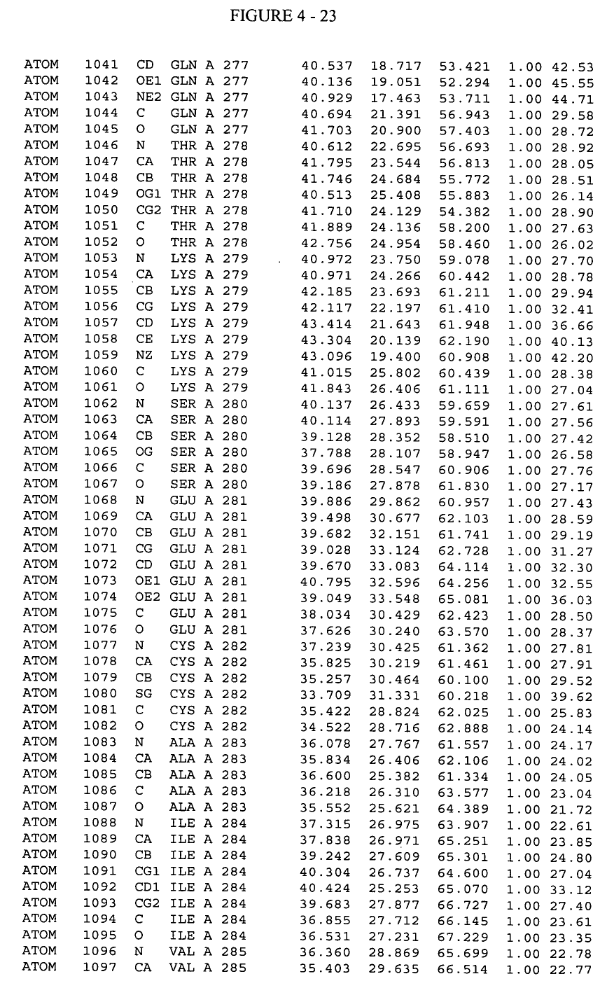

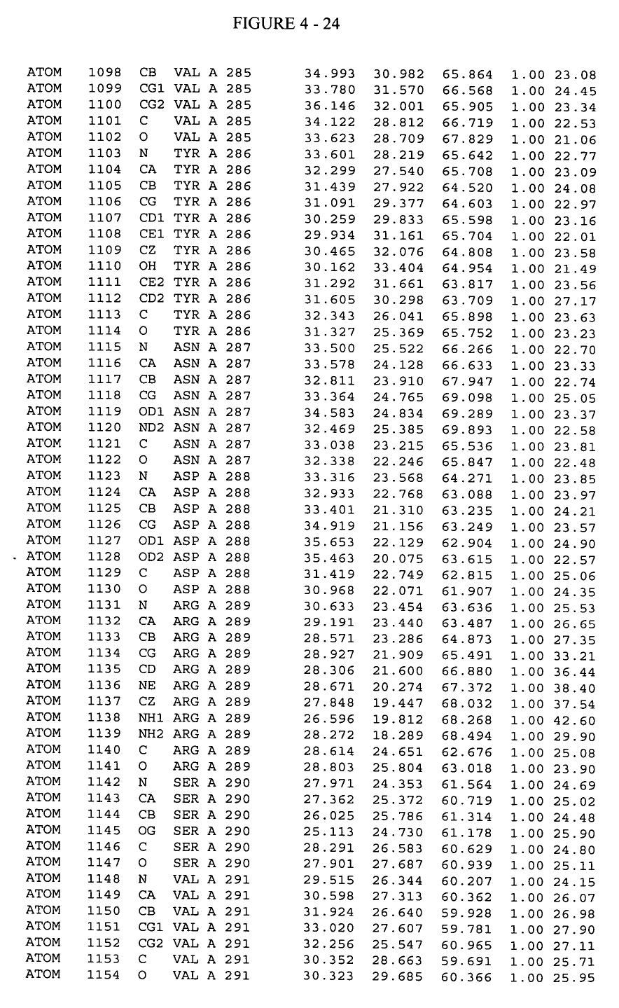

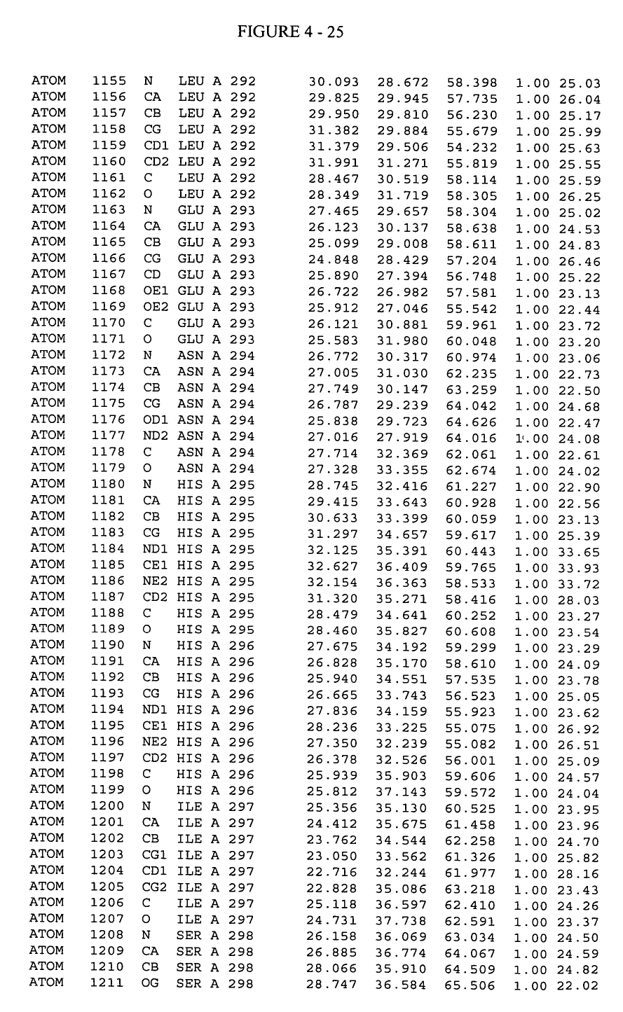

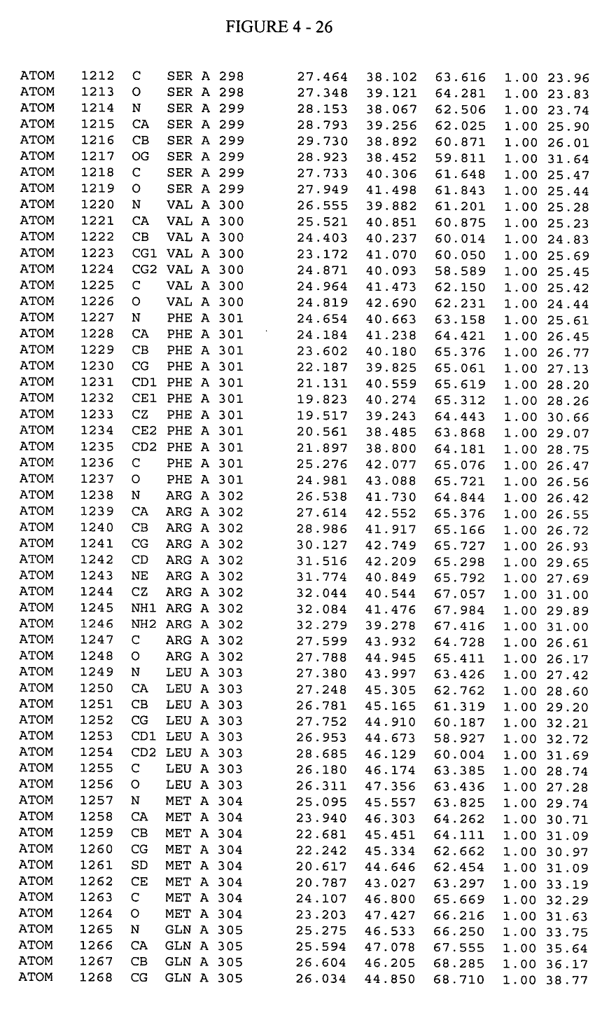

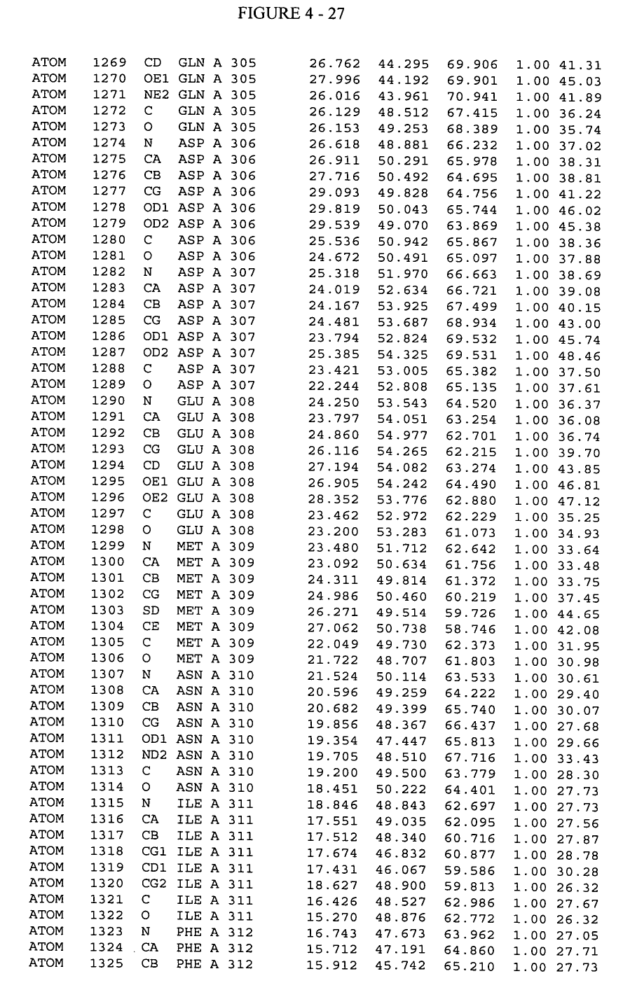

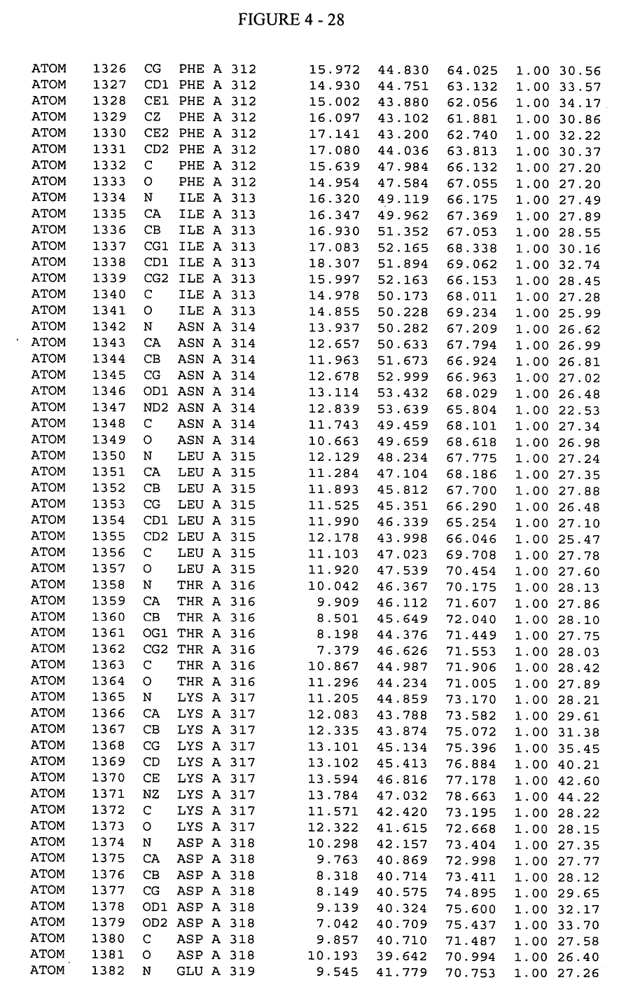

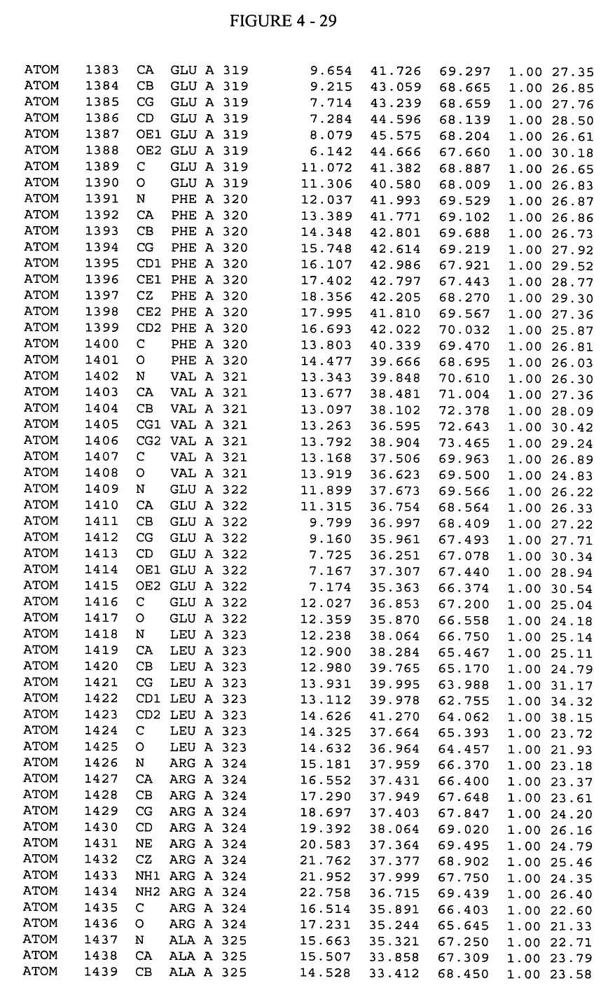

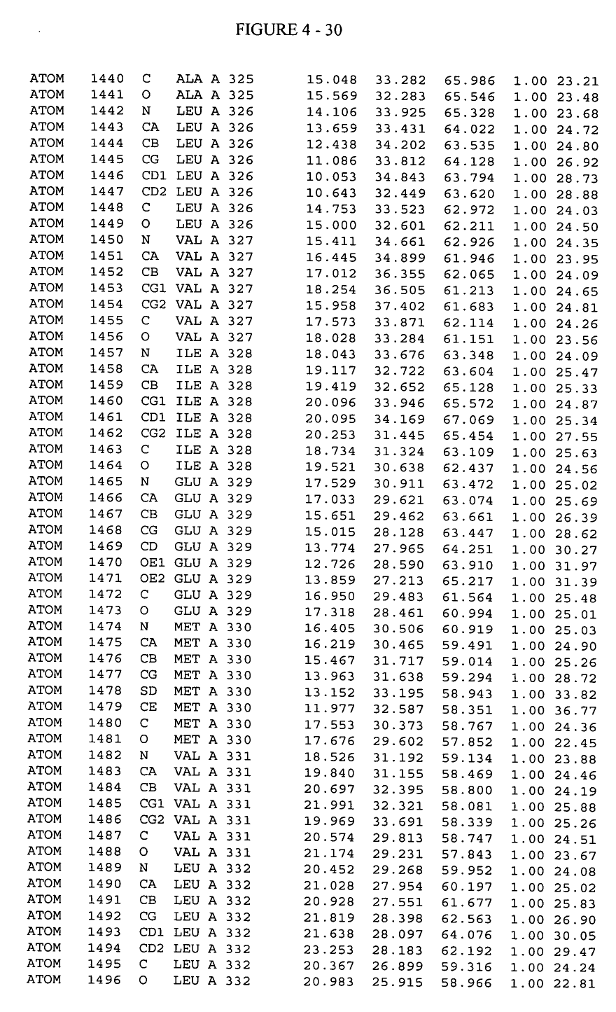

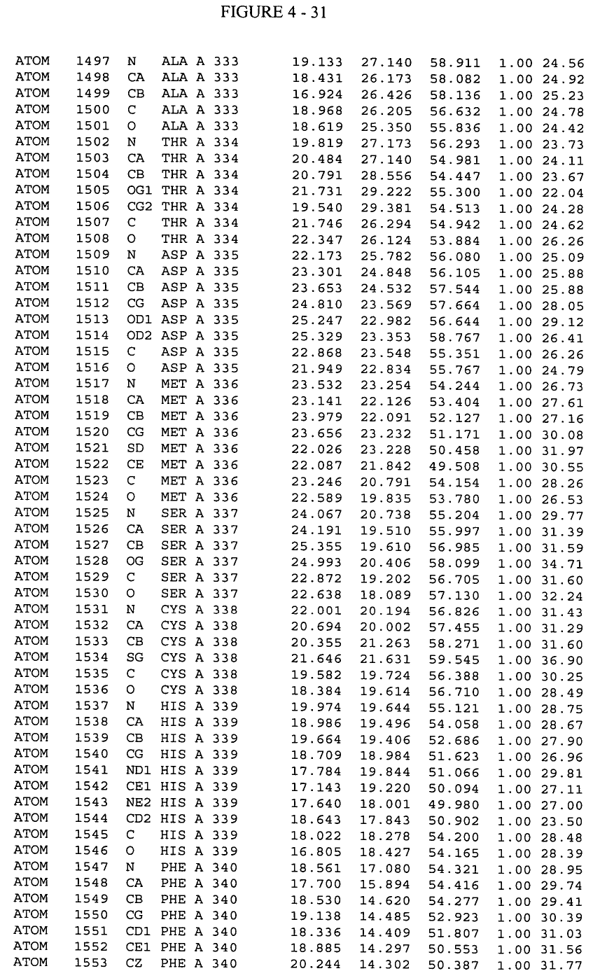

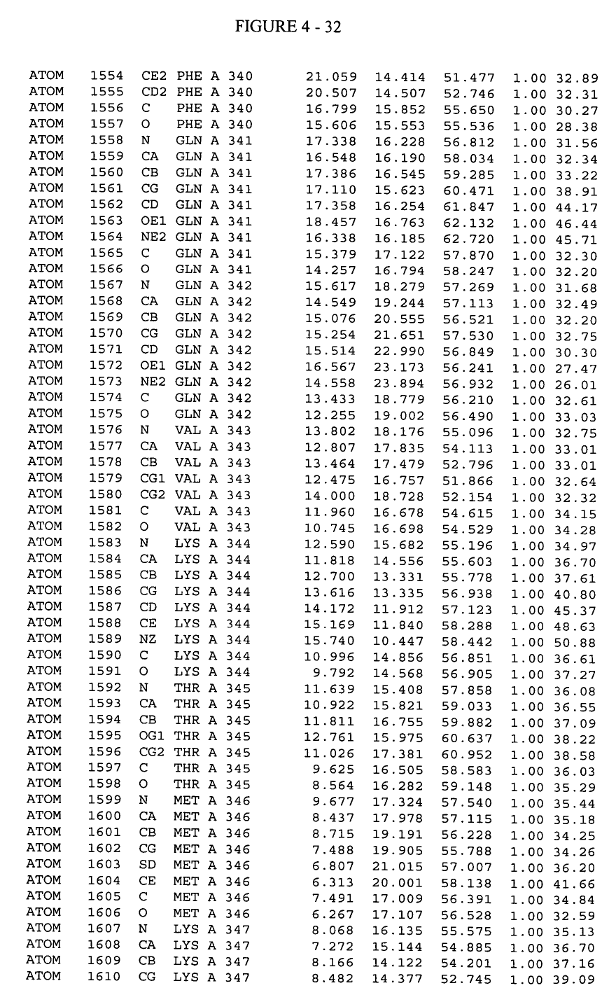

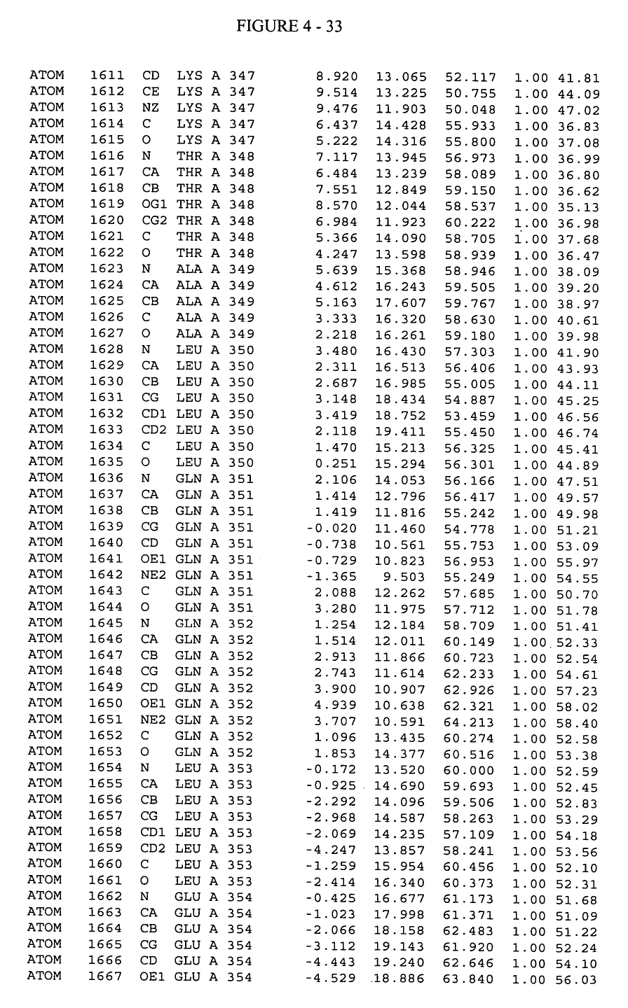

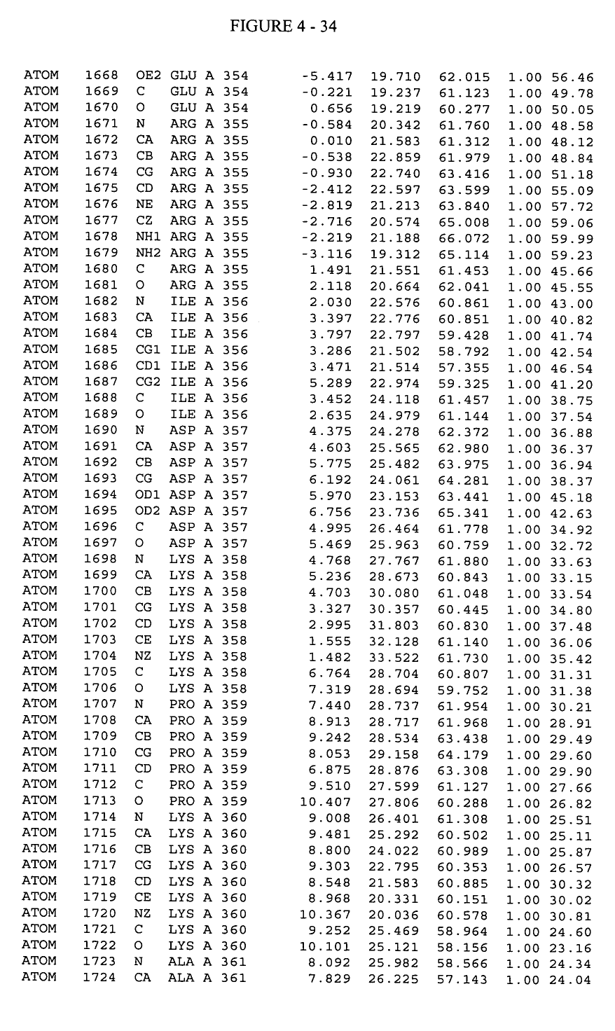

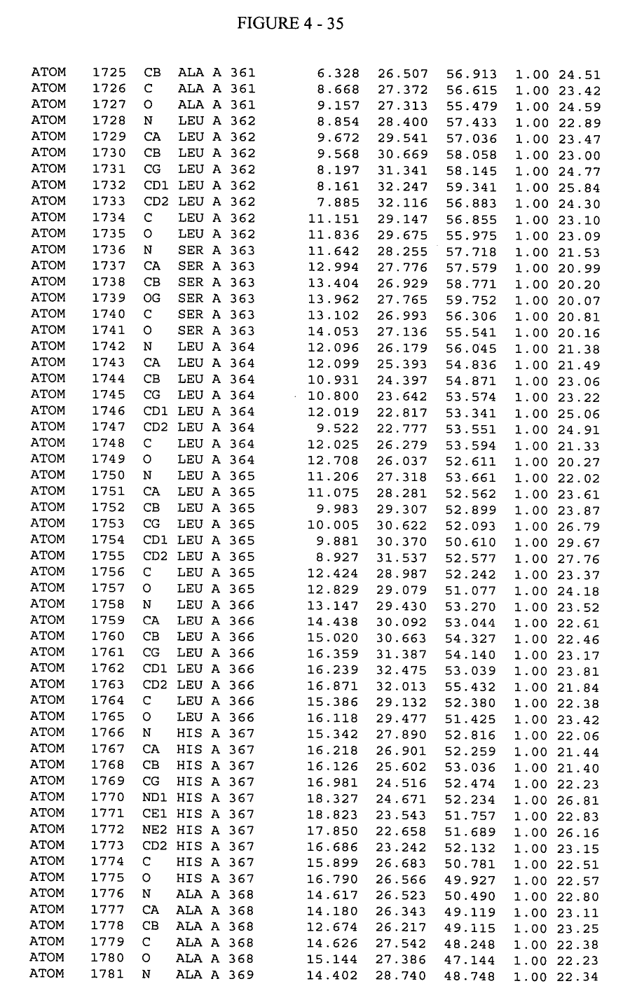

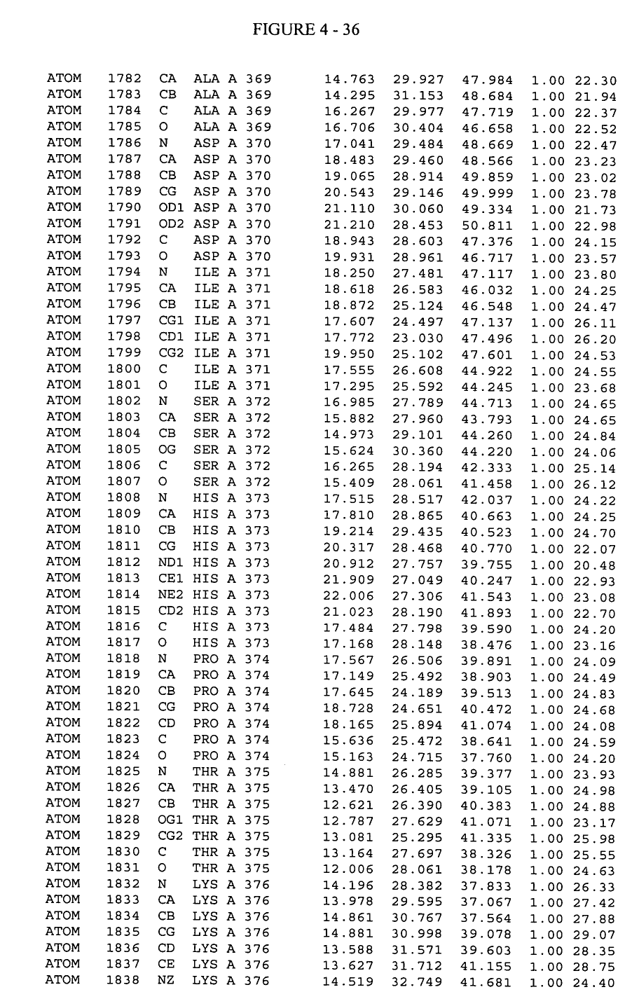

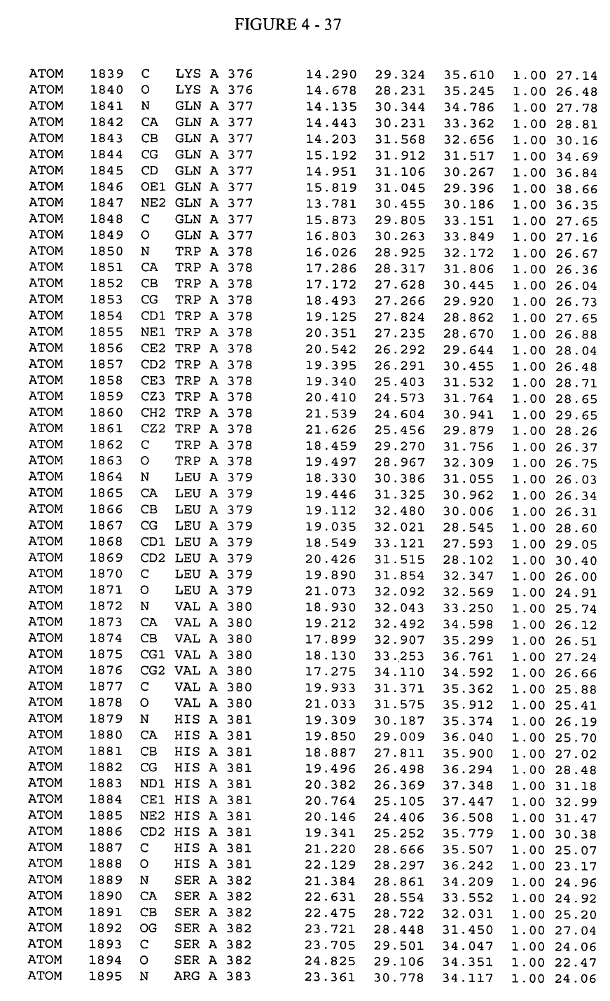

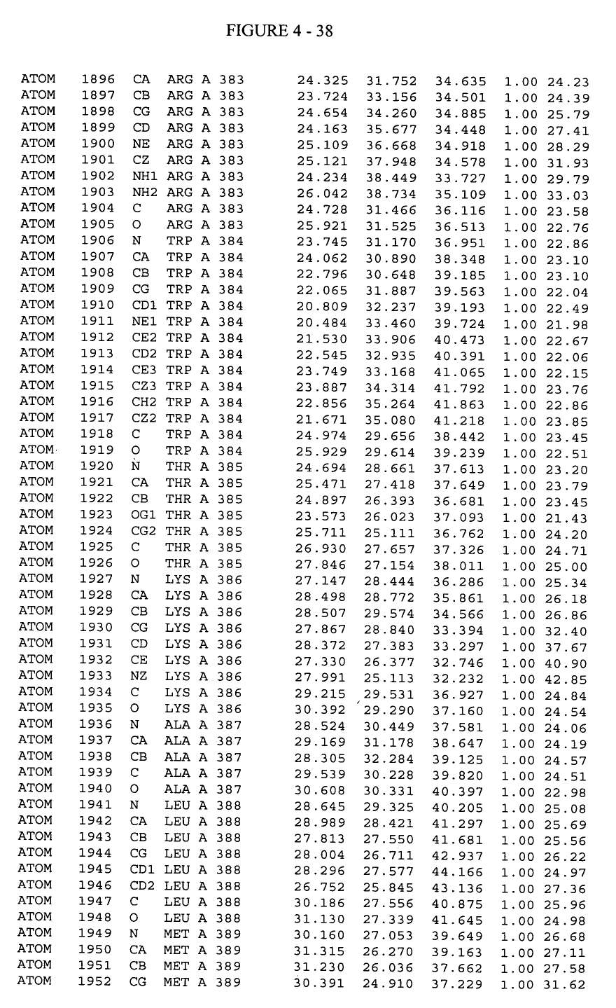

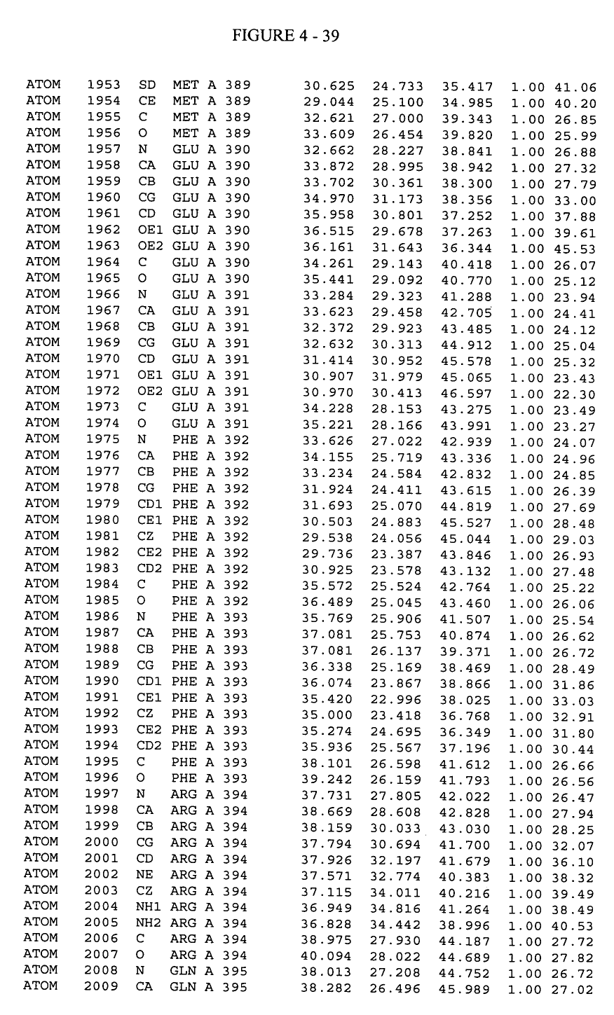

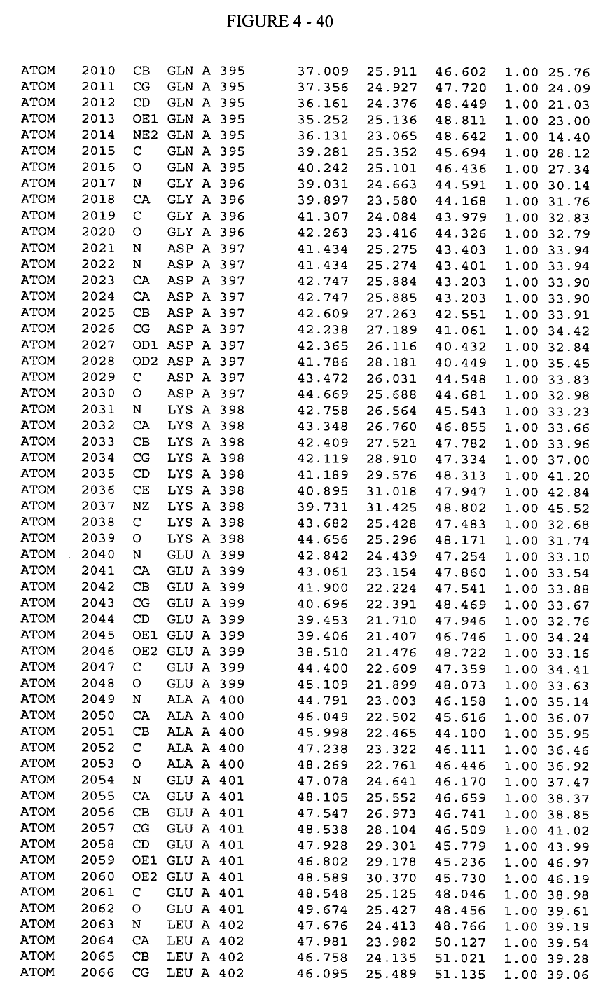

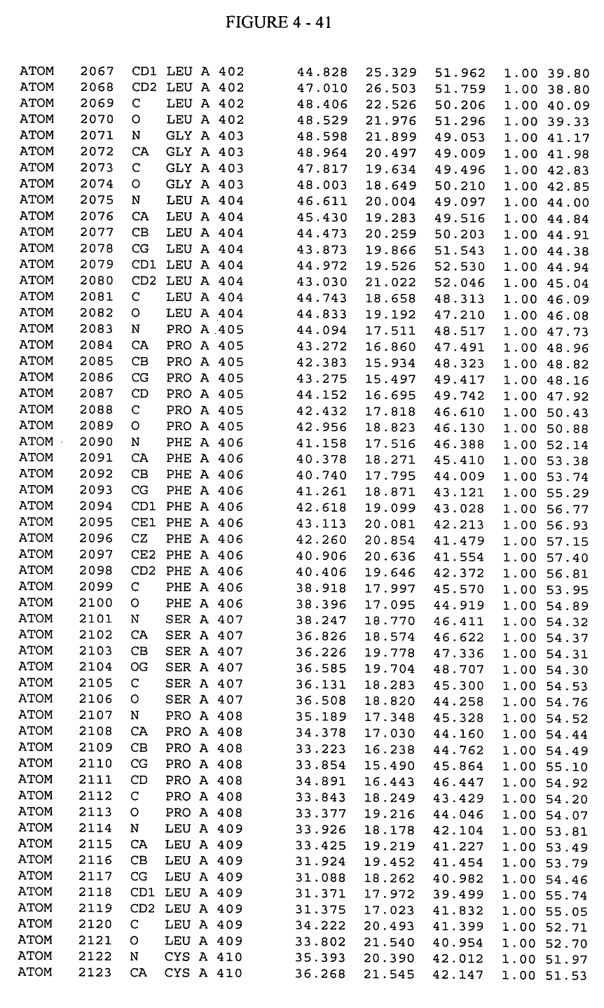

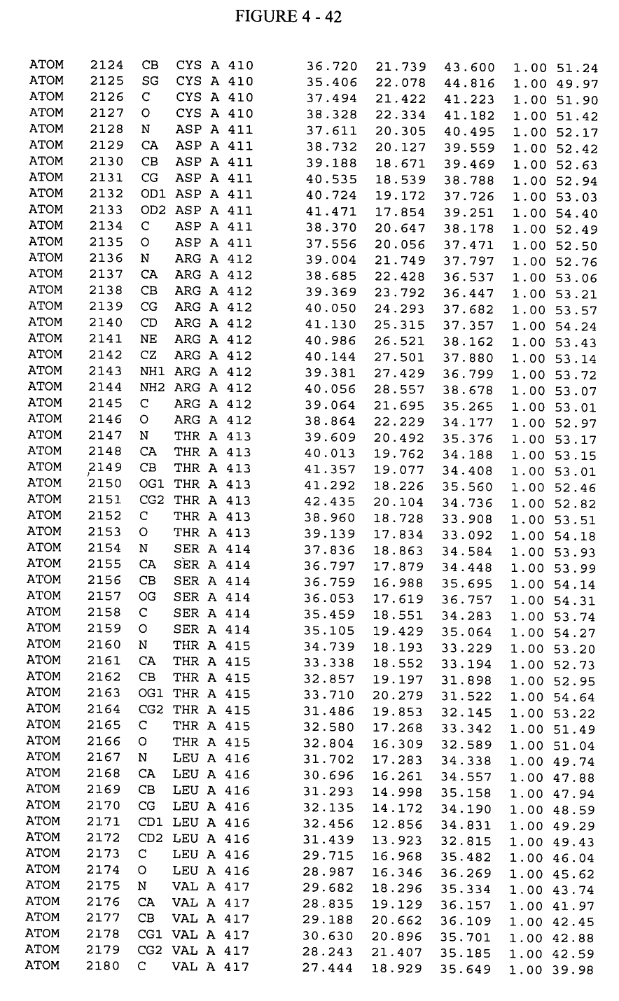

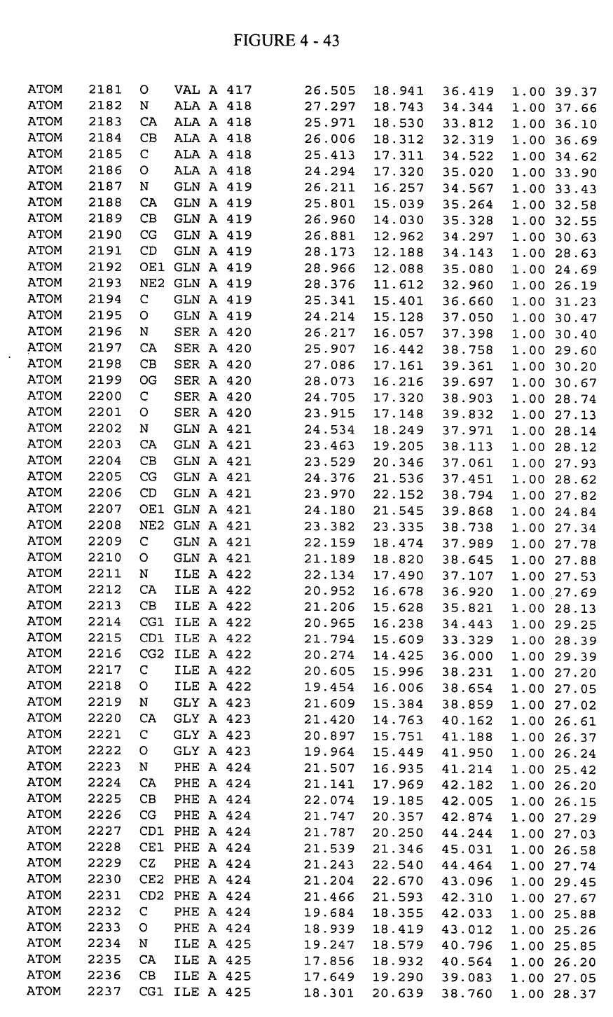

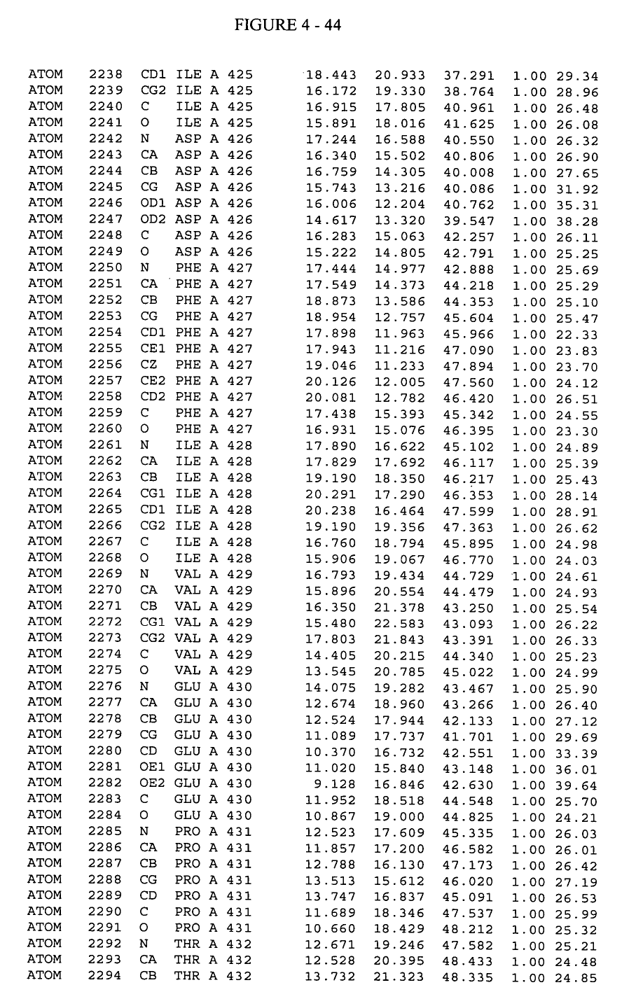

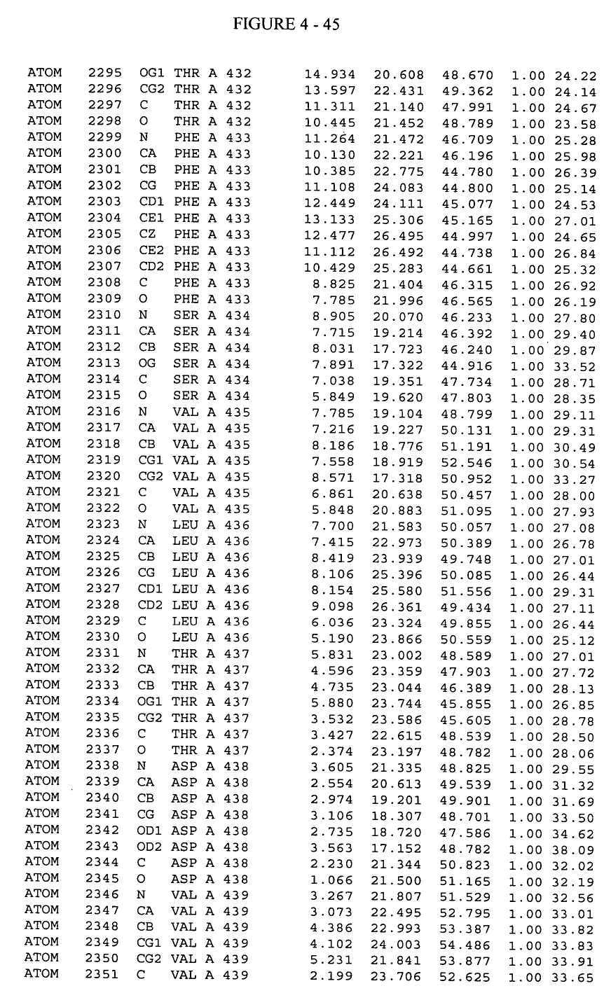

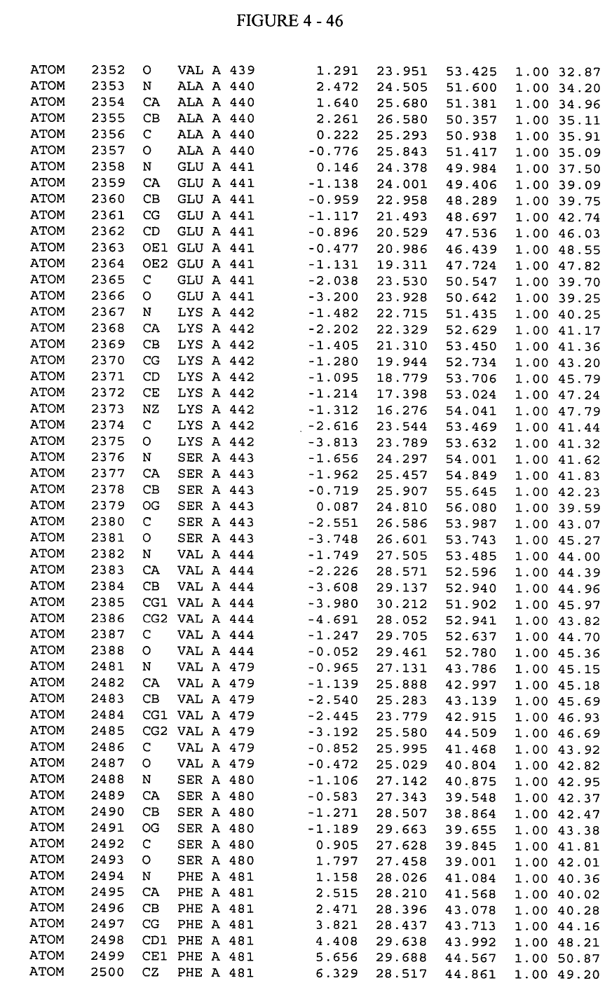

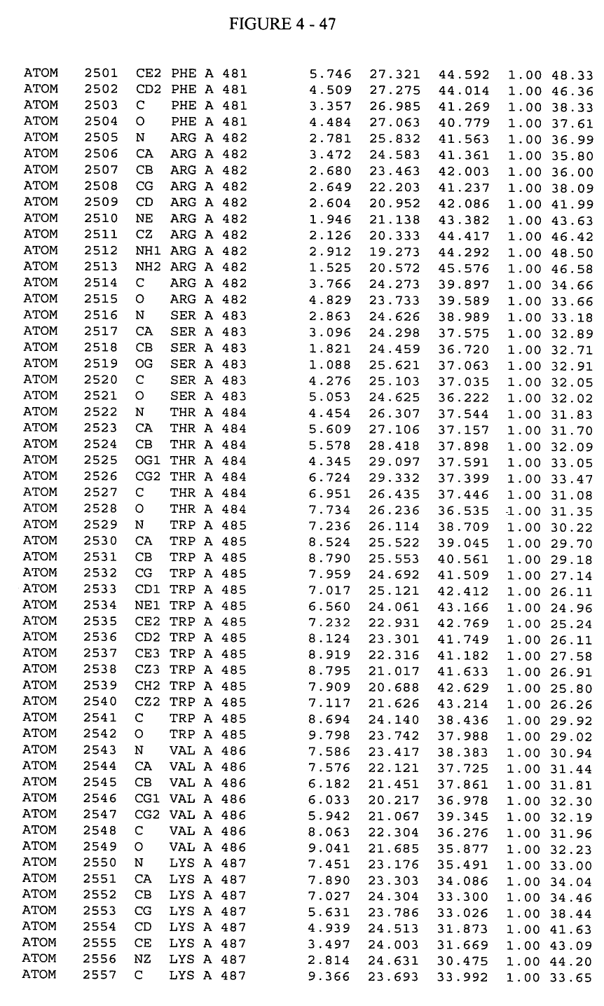

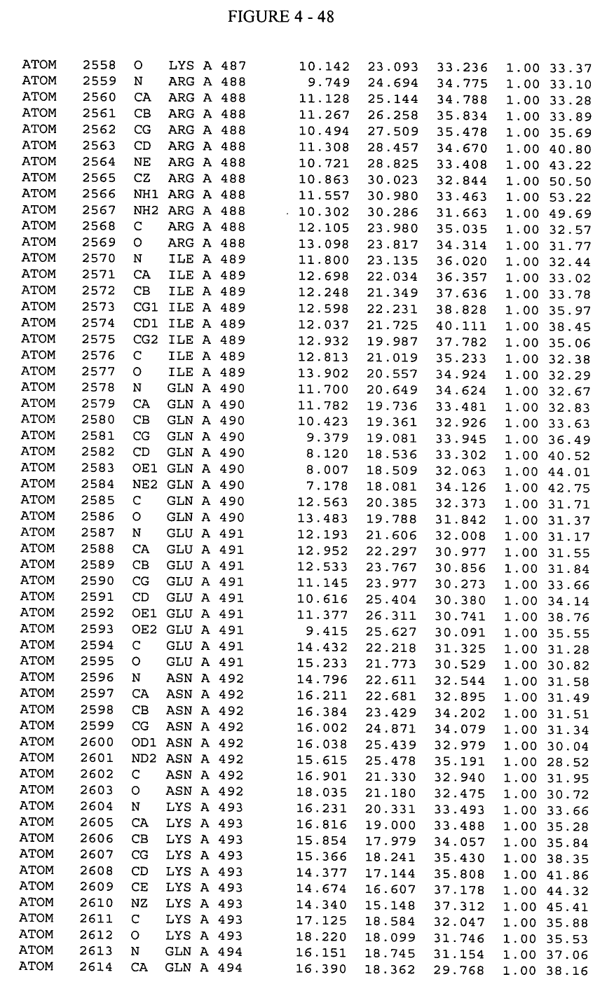

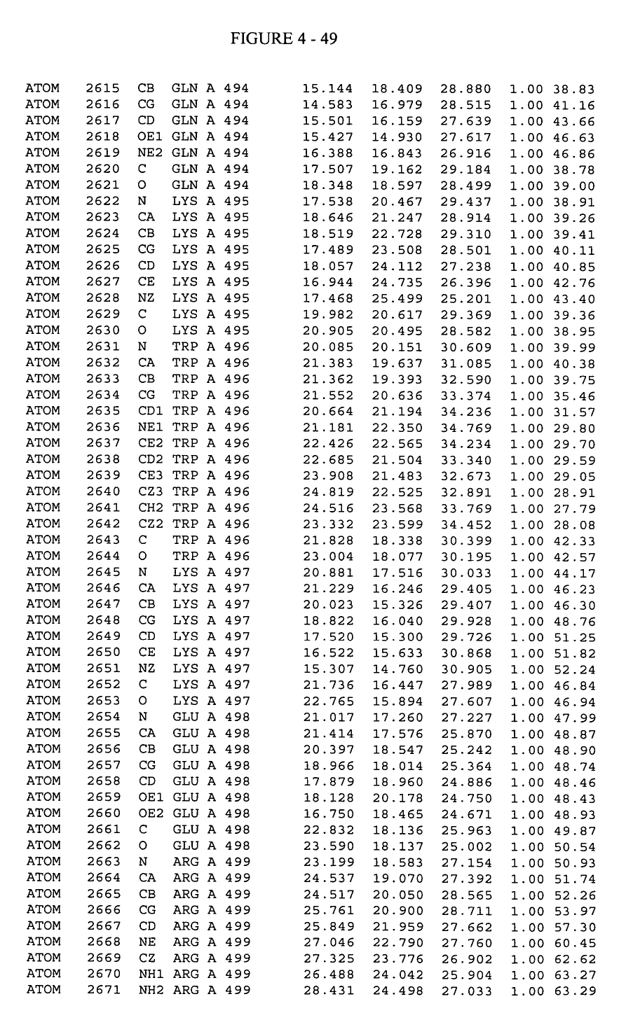

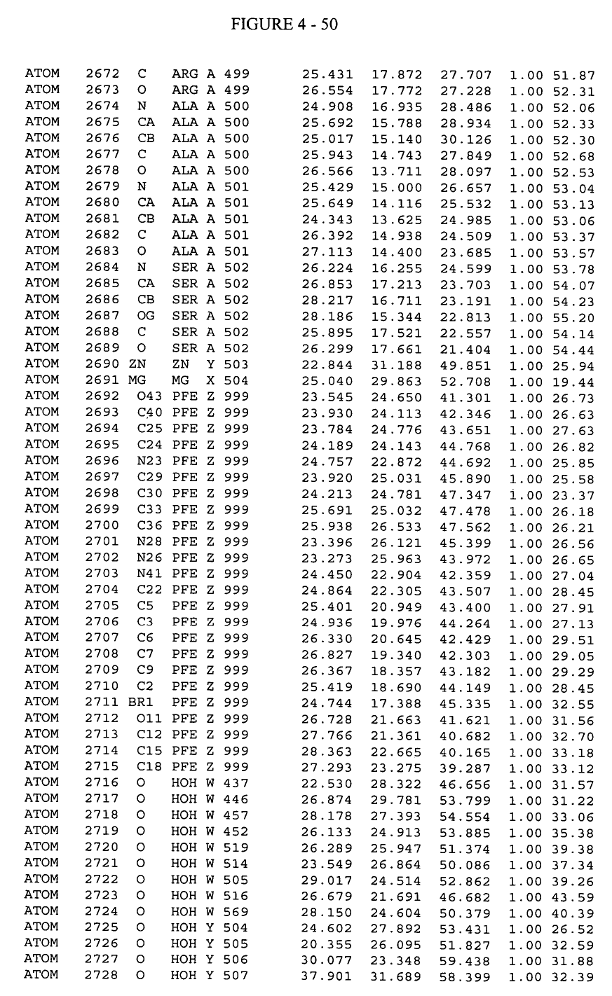

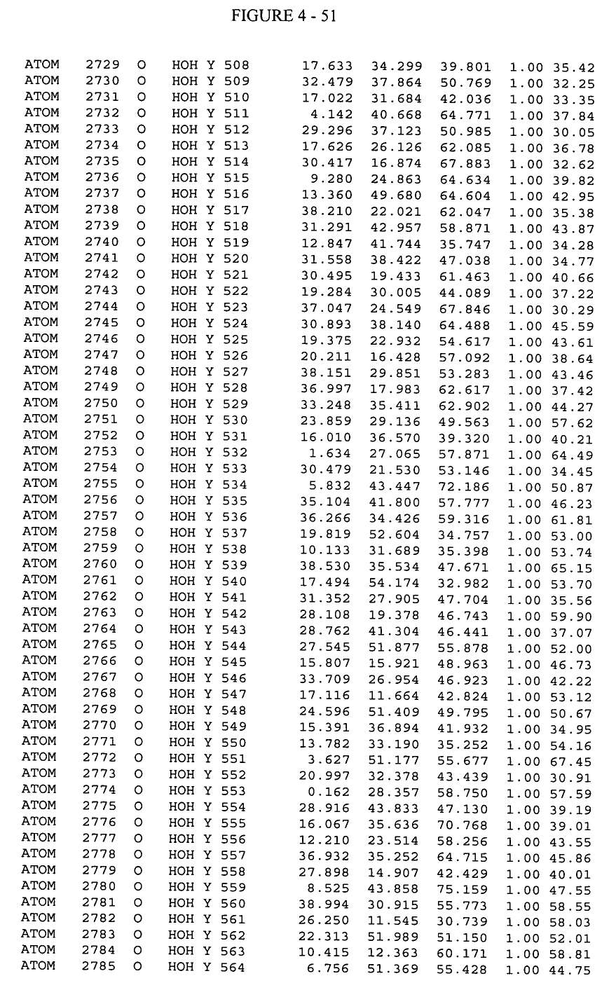

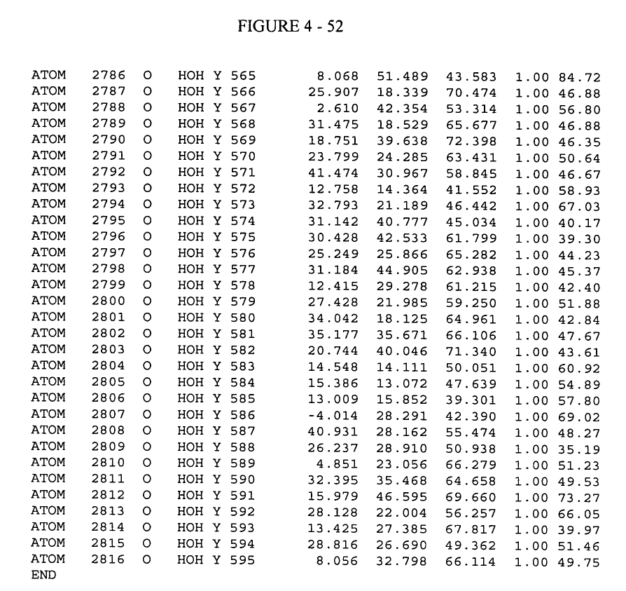

The term “homologue” as used herein refers to polypeptides having at least 50%, 45% or even 42%, amino acid sequence identity with PDE1B enzyme as described in SEQ ID NO:1 or 2 or any catalytic domain described herein. SEQ ID NO: 1 is the fuill-length amino acid sequence of the wild-type Human PDEIlB. SEQ ID NO: 2 is the amino acid sequence of the wild-type C-terminal catalytic domain of Human PDELB that was crystallized in the Examples. In certain preferred embodiments, the sequence identity is greater than about 60%, 65%, 70%, 75%, 80%, 85%, 90%, 95% or even 98%. Those of skill in the art understand that a set of structure coordinates determined by X-ray crystallography is not without standard error. As used herein, and for the purpose of this invention, the term “substantially similar atomic coordinates” or atomic coordinates that are “substantially similar” refers to any set of structure coordinates of PDE1B or PDE1B homologues, or PDE1B variants, polypeptide fragments, described by atomic coordinates that have a root mean square deviation for the atomic coordinates of protein backbone atoms (N, Ca, C, and O) of less than about 2.0, 1.7, 1.5, 1.2, 1.0, 0.7, 0.5, or even 0.2 Å when superimposed- using backbone atoms- of structure coordinates listed in FIG. 4. For the purpose of this invention structures that have substantially similar coordinates as those listed in FIG. 4 shall be considered identical to the coordinates listed in FIG. 4. The term “substantially similar” also applies an assembly of amino acid residues that may or may not form a contiguous polypeptide chain, but whose three dimensional arrangement of atomic coordinates have a root mean square deviation for the atomic coordinates of protein backbone atoms (N, Ca, C, and O), or the side chain atoms, of less than about 2.0, 1.7, 1.5, 1.2, 1.0, 0.7, 0.5, or even 0.2 Å when superimposed- using backbone atoms, or the side chain atoms- of the atomic coordinates of similar or the same amino acids from the coordinates listed in FIG. 4. To clarify further, but not intending to be limiting, an example of an assembly of amino acids may be the amino acid residues that form a binding site in an enzyme. These residues may have one or more intervening residues which are distant from the binding site, and therefore may minimally interact with a ligand in the binding sites. In such occurrences, the binding site may be defined for the purpose of structure based drug design as comprising only a handful of amino acid residues. For example in the case of PDE1B, amino acid residues His223, His373, Thr385, Leu388, Ser420, Gln421, and Phe424 of SEQ ED NO:1 are known to be near or at the binding site. Thus any molecular assembly that has a root mean square deviation from the atomic coordinates of the protein backbone atoms (N, Ca, C, and O), or the side chain atoms, of one or more of His223, His373, Thr385, Leu388, Ser420, Gln421, and Phe424 of SEQ ID NO:1, or any conservative substitutions thereof, of less than about 2.0, 1.7, 1.5, 1.2, 1.0, 0.7, 0.5, or even 0.2 Å when superimposed will be considered substantially similar to the coordinates listed in FIG. 4. “Substantially similar” atomic coordinates, for the purposes of this invention are considered identical to the coordinates, or portions thereof, listed in FIG. 4.

Those skilled in the art further understand that the coordinates listed in FIG. 4 or portions thereof may be transformed into a different set of coordinates using various mathematical algorithms without departing from the present invention. For example, the coordinates listed in FIG. 4, or portions thereof, may be transformed by algorithms which translate or rotate the atomic coordinates. Alternatively, molecular mechanics, molecular dynamics or ab initio algorithms may modify the atomic coordinates. Atomic coordinates generated from the coordinates listed in FIG. 4, or portions thereof, using any of the aforementioned algorithms shall be considered identical to the coordinates listed in FIG. 4.

The term “in silico” as used herein refers to experiments carried out using computer simulations. In certain embodiments, the in silico methods are molecular modeling methods wherein 3-dimensional models of macromolecules or ligands are generated. In other embodiments, the in silico methods comprise computationally assessing ligand binding interactions.

The term “modulate” as used herein refers to both upregulation (i.e., activation or stimulation, e.g., by agonizing or potentiating) and down-regulation (i.e., inhibition or suppression, e.g., by antagonizing, decreasing or inhibiting) of an activity.

The term “pharmacophore” as used herein refers to the ensemble of steric and electronic features of a particular structure that is necessary to ensure the optimal supramolecular interactions with a specific biological target structure and to trigger (or to block) its biological response. A pharmacophore may or may not represent a real molecule or a real association of functional groups. In certain embodiments, a pharmacophore is an abstract concept that accounts for the common molecular interaction capacities of a group of compounds towards their target structure. In certain other embodiments, the term can be considered as the largest common denominator shared by a set of active molecules. Pharmacophoric descriptors are used to define a pharmacophore, including H- bonding, hydrophobic and electrostatic interaction sites, defined by atoms, ring centers and virtual points. Accordingly, in the context of enzyme agonists, antagonists or ligands, a pharmacophore may represent an ensemble of steric and electronic factors which are necessary to insure supramolecular interactions with a specific biological target structure. As such, a pharmacophore may represent a template of chemical properties for an active site of a protein/enzyme—representing these properties' spatial relationship to one another—that theoretically defines a ligand that would bind to that site.

The term “precipitant” as used herein is includes any substance that, when added to a solution, causes a precipitate to form or crystals to grow. Examples of precipitants within the scope of this invention include, but are not limited to, alkali (e.g., Li, Na, or K), or alkaline earth metal (e.g., Mg, or Ca) salts, and transition (e.g., Mn, or Zn) metal salts. Common counterions to the metal ions include, but are not limited to, halides, phosphates, citrates and sulfates.

The term “prodrug” as used herein refers to drugs that, once administered, are chemically modified by metabolic processes in order to become pharmaceutically active. In certain embodiments the term also refers to any compound that undergoes biotransformation before exhibiting its pharmacological effects. Prodrugs can thus be viewed as drugs containing specialized non-toxic protective groups used in a transient manner to alter or to eliminate undesirable properties in the parent molecule.

The term “receptor” as used herein refers to a protein or a protein complex in or on a cell that specifically recognizes and binds to a compound acting as a molecular messenger (neurotransmitter, hormone, lymphokine, lectin, drug, etc.). In a broader sense, the term receptor is used interchangeably with any specific (as opposed to non- specific, such as binding to plasma proteins) drug binding site, also including nucleic acids such as DNA.

The term “recombinant protein” refers to a polypeptide which is produced by recombinant DNA techniques, wherein generally, DNA encoding a polypeptide is inserted into a suitable expression vector which is in turn used to transform a host cell to produce the polypeptide encoded by said DNA. This polypeptide may be one that is naturally expressed by the host cell, or it may be heterologous to the host cell, or the host cell may have been engineered to have lost the capability to express the polypeptide which is otherwise expressed in wild type forms of the host cell. The polypeptide may also be a fusion polypeptide. Moreover, the phrase “derived from”, with respect to a recombinant gene, is meant to include within the meaning of “recombinant protein” those proteins having an amino acid sequence of a native polypeptide, or an amino acid sequence similar thereto which is generated by mutations, including substitutions, deletions and truncation, of a naturally occurring form of the polypeptide.

As used herein, the term “selective PDE1B inhibitor” refers to a substance, for example an organic molecule that effectively inhibits an enzyme from the PDE1B family to a greater extent that may other PDE enzyme, particularly any enzyme from the PDE 1-9 families or any PDE11 enzyme. In one embodiment, a selective PDE1B inhibitor is a substance, for example, a small organic molecule having a Kᵢ for inhibition of PDE1B that is less than about one-half, one-fifth, or one-tenth the Kᵢ that the substance has for inhibition of any other PDE enzyme. In other words, the substance inhibits PDE1B activity to the same degree at a concentration of about one-half, one-fifth, one-tenth or less than the concentration required for any other PDE enzyme. In general a substance is considered to effectively inhibit PDE1B if it has an IC₅₀ or Ki of less than or about 10 μM, 1 μM, 500nM, 100 nM, 50 nM or even 10 nM.

As used herein the term “small molecules” refers to preferred drugs as they are orally available (unlike proteins which must be administered by injection or topically). Size of small molecules is generally under 1000 Daltons, but many estimates seem to range between 300 to 700 Daltons.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the polypeptide is disrupted.

The term “variant” in relation to the polypeptide sequence in SEQ ID NO:1 or SEQ ID NO:2 include any substitution of, variation of, modification of, replacement of, deletion of, or addition of one or more amino acids from or to the sequence providing a resultant polypeptide sequence for an enzyme having PDE1B activity. Preferably the variant, homologue, fragment or portion, of SEQ ID NO:1 or SEQ ID NO:2, comprises a polypeptide sequence of at least 5 contiguous amino acids, preferably at least 10 contiguous amino acids, preferably at least 15 contiguous amino acids, preferably at least 20 contiguous amino acids, preferably at least 25 contiguous amino acids, or preferably at least 30 contiguous amino acids.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

II. Clones and Expressions

The nucleotide sequence coding for a PDE1B polypeptide, or functional fragment, including the C-terminal peptide fragment of the catalytic domain of PDE1B protein, derivatives or analogs thereof, including a chimeric protein, thereof, can be inserted into an appropriate expression vector, i.e., a vector which contains the necessary elements for the transcription and translation of the inserted protein-coding sequence. The elements mentioned above are termed herein a “promoter.” Thus, the nucleic acid encoding a PDE1B polypeptide of the invention or a functional fragment comprising the C-terminal peptide fragment of the catalytic domain of PDE1B protein, derivatives or analogs thereof, is operationally associated with a promoter in an expression vector of the invention. In preferred embodiments, the expression vector contains the nucleotide sequence coding for the polypeptide comprising the amino acid sequence spanning amino acids Thr142 to Gln507 listed in SEQ ID NO:1. Both cDNA and genomic sequences can be cloned and expressed under the control of such regulatory sequences. An expression vector also preferably includes a replication origin. The necessary transcriptional and translational signals can be provided on a recombinant expression vector. As detailed below, all genetic manipulations described for the PDE1B gene in this section, may also be employed for genes encoding a functional fragment, including the C-terminal peptide fragment of the catalytic domain of the PDE1B protein, derivatives or analogs thereof, including a chimeric protein thereof.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant PDELB protein of the invention may be expressed chromosomally, after integration of the coding sequence by recombination. In this regard, any of a number of amplification systems may be used to achieve high levels of stable gene expression (See Sambrook et al., 1989, infra, the pertinent disclosure of which is incorporated by reference herein in its entirety).

A suitable cell for purposes of this invention is one into which the recombinant vector comprising the nucleic acid encoding PDE1B protein is cultured in an appropriate cell culture medium under conditions that provide for expression of PDE1B protein by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques, and in vivo recombination (genetic recombination).

Expression of PDE1B protein may be controlled by any promoter/enhancer element known in the art, but these regulatory elements must be functional in the host selected for expression.

Expression vectors containing a nucleic acid encoding a PDE1B protein of the invention can be identified by four general approaches: (1) PCR amplification of the desired plasmid DNA or specific MRNA, (2) nucleic acid hybridization, (3) presence or absence of selection marker gene functions, and (4) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., .beta.-galactosidase activity, thymidine kinase activity, resistance to antibiotics, transformation phenotype, occlusion body formation in baculovirus, etc.) caused by the insertion of foreign genes in the vector. In another example, if the nucleic acid encoding PDE1B protein is inserted within the “selection marker” gene sequence of the vector, recombinant vectors containing the PDE1B protein insert can be identified by the absence of the PDE1B protein gene function. In the fourth approach, recombinant expression vectors can be identified by assaying for the activity, biochemical, or immunological characteristics of the gene product expressed by the recombinant vector, provided that the expressed protein assumes a functionally active conformation.

A wide variety of host/expression vector combinations may be employed in expressing the DNA sequences of this invention. Useful expression vectors, for example, may consist of segments of chromosomal, nonchromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., 1988, Gene 67:31-40), pMB9 and their derivatives, plasmids such as RP4; phage DNAS, e.g., the numerous derivatives of phage lambda., e.g., NM989, and other phage DNA, e.g., M13 and filamentous single stranded phage DNA; yeast plasmids such as the 2.mu. plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamrH1 cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamHI recognition site begins with the initiation codon; Summers), pAc701 and pAc702 (same as pAc700, with different reading frames), pAc360 (Bam-H1 cloning site 36 base pairs downstream of a polyhedron initiation codon; Invitrogen(195)), and pBlueBacHisA, B, C (three different reading frames, with BamH1, BglII, PstI, NcoI, and HindIlI cloning site, an N-terminal peptide for ProBond purification, and blue/white recombinant screening of plaques.

Exemplary mammalian expression vectors for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, Smal, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, Smal, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (Kpnl, PvuI, NheI, HindIII, NotI, XhoI, SfiI, BamH1 cloning site, inducible metallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindlIl, BstXI, NotI, SbaI, and ApaI cloning site, G418 selection; Invitrogen), pRc/RSV (HindIII, SpeI, BstXI, NotI, XhaI cloning site, G418 selection; Invitrogen), and others. Vaccinia virus mammalian expression vectors (see, Kaufinan, 1991, supra) for use according to the invention include but are not limited to pSC11 (Smal cloning site, TK- and .beta.-gal selection), pMJ601 (SalI, SmaI, AflI, NarI, BspMII, BamHI, ApaI, NheI, SacII, KpnI, and HindIII cloning site; TK- and .beta.-gal selection), and pTKgptF1S (EcoRI, PstI, SalI, AccI, HindIII, SbaI, BamHI, and Hpa cloning site, TK or XPRT selection)

Yeast expression systems can also be used according to the invention to express PDE1B polypeptide. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, Shol, NotI, BstXI, EcoRI, BamH1, SacI, KpnI, and HindIII cloning site, N-terminal peptide purified with ProBond resin and cleaved with enterokinase; Invitrogen), to mention just two, can be employed according to the present invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

Vectors can be introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

III. Crystal and Space Groups

X-ray structure coordinates define a unique configuration of points in space. Those skilled in the art understand that a set of structure coordinates for a protein or a protein/ligand complex, or a portion thereof, define a relative set of points that, in turn, define a configuration in three dimensions. A similar or identical configuration can be defined by an entirely different set of coordinates, provided the distances and angles between atomic coordinates remain essentially the same. In addition, a scalable configuration of points can be defined by increasing or decreasing the distances between coordinates by a scalar factor while keeping the angles essentially the same. One of ordinary skill in the art would recognize that solving atomic coordinates of crystal structures of proteins such as PDE1B requires a stable, long-lasting source of high-quality protein.

One aspect of the present invention relates to a crystalline composition comprising a polypeptide with an amino acid sequence spanning amino acids Thr142 to Gln507 listed in SEQ ID NO:1.

In one embodiment, the present invention discloses a crystalline PDE1B molecule comprising a polypeptide with an amino acid sequence spanning amino acids Thr142 to Gln507 listed in SEQ ID NO:1 complexed with one or more ligands. In one embodiment, the crystallized complex is characterized by the structural coordinates listed in FIG. 4, or portions thereof. In certain embodiments, the atoms of the ligand are within about 4, 7, or 10 angstroms of one or more PDE1B amino acids in SEQ ID NO: 1 preferably selected from His223, His373, Thr385, Leu388, Ser420, Gln421, and Phe424. One embodiment of the crystallized complex is characterized as belonging to the P4₃2₁2 space group and has unit cell dimensions a=87.47, b=87.47, c=135.03 Å, α=β=γ90.0°. This embodiment is encompassed by the structural coordinates of FIG. 4. The ligand may be a small molecule which binds to a PDE1B catalytic domain defined by SEQ ID NO:2, or portions thereof, with a Kᵢ of less than about 10 μM, 1 μM, 500 nM, 100 nM, 50 nM, or even 10 nM. In a certain embodiment, the ligand is Compound 109 (5-(5-bromo-2-propoxy-phenyl)-3 -propyl- 1,6-dihydro-pyrazolo[4,3-d]pyrimidin-7-one). One of ordinary skill in the art will recognize that other ligand(s) may be used without departing from the present invention. In certain embodiments, the ligand is a substrate or substrate analog of PDE1B. In certain embodiments, the ligand(s) may be a competitive or uncompetitive inhibitor of PDE1B. In certain embodiments, the ligand is a covalent inhibitor of PDE1B.

IV. Structurally Equivalent Crystal Structures

Various computational methods can be used to determine whether a molecule or a binding pocket portion thereof is “structurally equivalent,” defined in terms of its three-dimensional structure, to all or part of PDE1B or its binding pocket(s). Such methods may be carried out in current software applications, such as the molecular similarity application of QUANTA (Accelrys Inc., San Diego, Calif.).

The molecular similarity application permits comparisons between different structures, different conformations of the same structure, and different parts of the same structure. The procedure used in molecular similarity to compare structures is divided into four steps: (1) load the structures to be compared into a computer; (2) optionally define the atom equivalences in these structures; (3) perform a fitting operation; and (4) analyze the results.

Each structure is identified by a name. One structure is identified as the target (i.e., the fixed structure); all remaining structures are working structures (i.e., moving structures). Since atom equivalency within molecular similarity applications is defined by user input, for the purpose of this invention equivalent atoms are defined as protein backbone atoms (N, Cα, C, and O) for all conserved residues between the two structures being compared. A conserved residue is defined as a residue that is structurally or functionally equivalent (See Table 4 infra). In certain embodiments rigid fitting operations are considered. In other embodiments, flexible fitting operations may be considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atoms is an absolute minimum. This number, given in angstroms, is reported by the molecular similarity application.

For the purpose of this invention, any molecule or molecular complex or binding pocket thereof, or any portion thereof, that has a root mean square deviation of conserved residue backbone atoms (N, Cα, C, and O) of less than about 2.0, 1.7, 1.5, 1.25, 1.0, 0.7, 0.5, or even 0.2 Å, when superimposed on the relevant backbone atoms described by the reference structure coordinates listed in FIG. 4, is considered structurally equivalent” to the reference molecule. That is to say, the crystal structures of those portions of the two molecules are substantially identical, within acceptable error. Particularly preferred structurally equivalent molecules or molecular complexes are those that are defined by the entire set of structural coordinates listed in FIG. 4, plus or minus a root mean square deviation from the conserved backbone atoms of those amino acids of not more than about 2.0 Å. More preferably, the root mean square deviation is less than about 1.0 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of PDE1B or a binding pocket portion thereof, as defined by the structural coordinates of PDE1B described herein.

V. Description of Crystal Structure and Binding Site

The refined x-ray coordinates of the catalytic domain of PDE1B (SEQ ID NO:2), complexed with Compound 109, Zn²⁺, Mg²⁺, and 101 water molecules are as listed in FIG. 4.

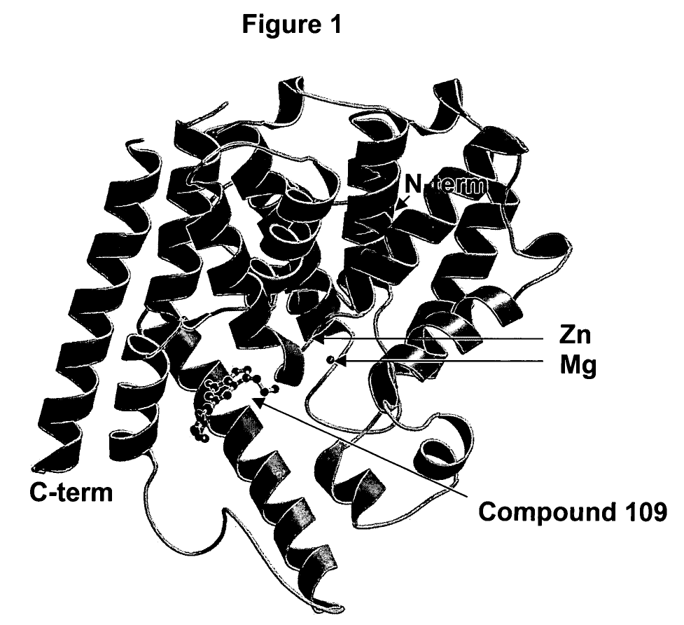

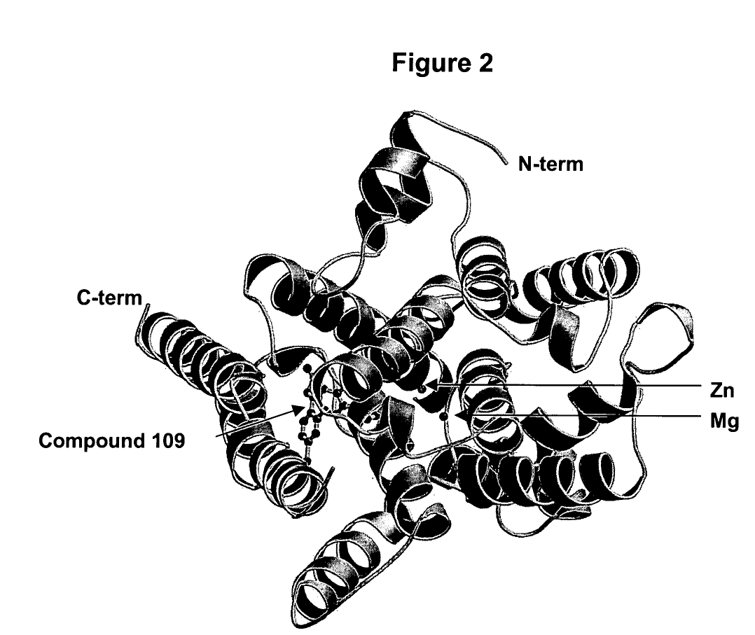

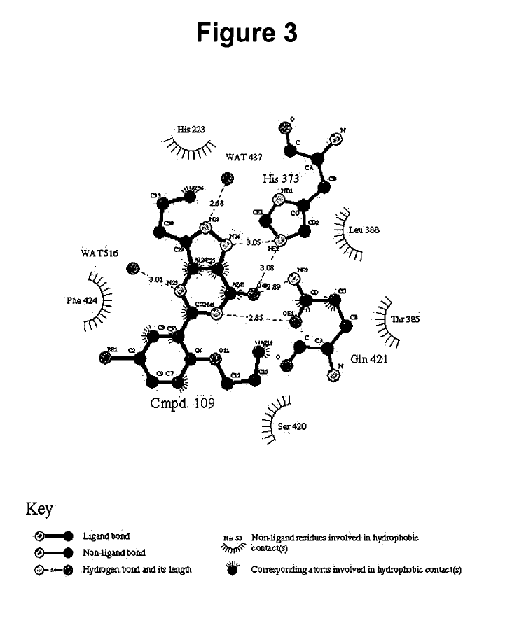

Two orthogonal views of the molecule are shown in FIG. 1 and FIG. 2, and details of the interactions of the inhibitor with the protein are shown in FIG. 3.

The structure is composed of a single domain of sixteen α helices and four 3₁₀ helices arranged in a compact fold (FIG. 1 and 2). The numbering of the helices is as shown below. We have followed the numbering convention established by Xu et al., Science, 288:1822-25 (2000), and the start and end points of the helices are determined according to Kabsch and Sander, Biopolymers, 22(12): 2577-637 (1983).

| | |
|---|---|
| αhelices | 3₁₀ helices |

| | | | |
|---|---|---|---|
| H1 | 152-158 | A1 | 244-247 |
| H2 | 168-174 | A2 | 305-309 |
| H3/4 | 178-197 | A3 | 334-337 |
| H5 | 201-216 | A4 | 373-375 |
| H6 | 225-242 | | |
| H7 | 250-262 | | |
| H8 | 272-277 | | |
| H9 | 281-286 | | |
| H10 | 291-303 | | |
| H11 | 317-332 | | |
| H12 | 339-350 | | |
| H13 | 358-370 | | |
| H14 | 378-401 | | |
| H15 | 417-442 | | |
| H16 | 480-501 | | |

Within the overall fold, three sub-domains can also be defined. Residues 148-270 (H1-H7) form the first sub-domain, 271-337 (H8-H11) form the second sub-domain, and 338-502 (H12-H16) form the third sub-domain. No electron density is observed for residues 445-478, which probably comprise a flexible loop between Helices 15 and 16. Sequence alignment of the PDE gene family shows that this flexible loop between Helices 15 and 16 is an insertion in the PDE1 sequence, unique to PDE1.

Two metal ions are seen in the catalytic site. The first is determined to be Zn²⁺, by analogy with PDE4b, and from an analysis of its coordination geometry. The metal is coordinated by His227 (Nε2-Zn 2.1 Å), His263 (Nε2-Zn 2.1 Å), Asp370 (Oδ2-Zn 2.1 Å), Asp264 (Oδ2-Zn 2.1 Å) and a water molecule (O-Zn 2.3 Å). These residues are completely conserved across the PDE gene family. The second metal ion is coordinated to Asp264 (Oδ2-Zn 2.1 Å) and to a water network that stabilizes the metal environment. Due to the coordination geometry and the relative observed electron density, this second metal ion has been refined as a Mg²⁺ in accordance with a similar observation in the PDE4 structure (Xu et al., Science, 288:1822-25 (2000)).

One molecule of the ligand, compound 109, is seen bound within the active site. The active site lies mainly within the third subdomain and is bounded one side by helices H15, H14, the C-terminus of H13 and the 3₁₀ helix A4, and on the other side by C-terminus of H5, the N-terminus of H6 and the loop region in between H5 and H6. Protein-ligand interactions are shown schematically in FIG. 3.

Accordingly, the present invention provides a molecule or molecular complex that includes at least a portion of a PDE1B and/or substrate binding pocket. In one embodiment, the PDE1B binding pocket includes the amino acids listed in Table 1, preferably the amino acids listed in Table 2, and more preferably the amino acids listed in Table 3, the binding pocket being defined by a set of points having a root mean square deviation of less than about 2.0, 1.7, 1.5, 1.2, 1.0, 0.7, 0.5, or even 0.2 Å, from points representing the backbone atoms of the amino acids in Tables 1-3. In another embodiment, the PDE1B substrate binding pocket includes the amino acids selected from His223, His373, Thr385, Leu388, Ser420, Gln421, and Phe424 from SEQ ID NO:1

TABLE 1
Residues near the binding pocket in PDE1B catalytic domain. Identified
residues are 10 Å away from Compound 109

| | | | | | | | |
|---|---|---|---|---|---|---|---|
| TYR222 | HIS223 | ASN224 | HIS227 | ASP230 | VAL231 | HIS263 | ASP264 |
| HIS267 | GLY269 | THR270 | THR271 | ASN272 | LEU292 | GLU293 | HIS296 |
| THR334 | ASP335 | MET336 | SER337 | HIS339 | PHE340 | HIS367 | ALA368 |
| ALA369 | ASP370 | ILE371 | SER372 | HIS373 | PRO374 | THR375 | VAL380 |
| HIS381 | SER382 | ARG383 | TRP384 | THR385 | LYS386 | ALA387 | LEU388 |
| MET389 | GLU390 | GLU391 | PHE392 | PHE393 | GLN395 | SER407 | PRO408 |
| LEU409 | CYS410 | ASP411 | SER414 | THR415 | LEU416 | VAL417 | ALA418 |
| GLN419 | SER420 | GLN421 | ILE422 | GLY423 | PHE424 | ILE425 | ASP426 |
| PHE427 | ILE428 | VAL429 | TRP496 | | | | |

TABLE 2
Residues near the binding pocket in PDE1B catalytic domain. Identified
residues are 7 Å away from compound 109

| | | | | | | | |
|---|---|---|---|---|---|---|---|
| TYR222 | HIS223 | HIS227 | ASP264 | HIS267 | THR271 | THR334 | MET336 |
| ASP370 | ILE371 | SER372 | HIS373 | PRO374 | HIS381 | TRP384 | THR385 |
| LYS386 | LEU388 | MET389 | GLU391 | PHE392 | PRO408 | LEU409 | LEU416 |
| VAL417 | ALA418 | SER420 | GLN421 | GLY423 | PHE424 | ILE425 | PHE427 |
| ILE428 | VAL429 | TRP496 | | | | | |

TABLE 3
Residues near the binding pocket in PDE1B catalytic domain. Identified
residues are 4 Å away from compound 109

| | | | | | | | | |
|---|---|---|---|---|---|---|---|---|
| TYR222 | HIS223 | HIS373 | THR385 | LEU388 | PHE392 | SER420 | GLN421 | PHE424 |

VI. Isolated Polypeptide and Variants

One embodiment of the invention describes an isolated polypeptide consisting of a portion of PDELB which functions as the binding site when folded in the proper 3-D orientation. One embodiment is an isolated polypeptide comprising a portion of PDE1B, wherein the portion starts at about amino acid residue T142, and ends at about amino acid residue Q507 as described in SEQ ID NO:1, or a sequence that is at least 70%, 75%, 80%, 85%, 90%, 95%, or 98% homologous to a polypeptide with an amino acid sequence spanning amino acids Thr142 to Gln507 listed in SEQ ID NO:1.

One embodiment of the invention comprises crystalline compositions comprising variants of PDE1B. Variants of the present invention may have an amino acid sequence that is different by one or more amino acid substitutions to the sequence disclosed in SEQ ID NO:1 or SEQ ID NO:2. Embodiments which comprise amino acid deletions and/or additions are also contemplated. The variant may have conservative changes (amino acid similarity), wherein a substituted amino acid has similar structural or chemical properties, for example, the replacement of leucine with isoleucine. Guidance in determining which and how many amino acid residues may be substituted, inserted, or deleted without adversely affecting biological or proposed pharmacological activity may be reasonably inferred in view of this disclosure, and may further be found using computer programs well known-in the art, for example, DNAStar® software.

Amino acid substitutions may be made, for instance, on the basis of similarity in polarity, charge, solubility, hydrophobicity, hydrophilicity, and/or the amphipathic nature of the residues as long as a biological and/or pharmacological activity of the native molecule is retained.

Negatively charged amino acids include aspartic acid and glutamic acid; positively charged amino acids include lysine and arginine; amino acids, with uncharged polar head groups having similar hydrophilicity values include leucine, isoleucine, and valine; amino acids with aliphatic head groups include glycine, alanine; asparagine, glutamine, serine; and amino acids with aromatic side chains include threonine, phenylalanine, and tyrosine.

Examples of conservative substitutions are set forth in Table 4 as follows:

TABLE 4

| Original Residue | Example conservative substitutions |
|---|---|
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; Ile; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

“Homology” is a measure of the identity of nucleotide sequences or amino 5 acid sequences. In order to characterize the homology, subject sequences are aligned so that the highest percentage homology (match) is obtained, after introducing gaps, if necessary, to achieve maximum percent homology. N- or C-terminal extensions shall not be construed as affecting homology. “Identity” per se has an art-recognized meaning and can be calculated using published techniques. Computer program methods to determine identity between two sequences, for example, include DNAStar®) software (DNAStar Inc. Madison, Wis.); the GCG® program package (Devereux, J., et al. Nucleic Acids Research (1984) 12(1): 387); BLASTP, BLASTN, FASTA (Atschul, S. F. et al., J. Molec Biol (1990) 215: 403). Homology (identity) as defined herein is determined conventionally using the well-known computer program, BESTFIT® (Wisconsin Sequence Analysis Package, Version 8 for Unix, Genetics Computer Group, University Research Park, 575 Science Drive, Madison, Wis., 53711). When using BESTFIT® or any other sequence alignment program (such as the Clustal algorithm from MegAlign software (DNAStar®) to determine whether a particular sequence is, for example, about 90% homologous to a reference sequence, according to the present invention, the parameters are set such that the percentage of identity is calculated over the full length of the reference nucleotide sequence or amino acid sequence and that gaps in homology of up to about 90% of the total number of nucleotides in the reference sequence are allowed.

Ninety percent of homology is therefore determined, for example, using the BESTFIT® program with parameters set such that the percentage of identity is calculated over the full length of the reference sequence, e.g., SEQ ID NO:1, and wherein up to 10% of the amino acids in the reference sequence may be substituted with another amino acid. Percent homologies are likewise determined, for example, to identify preferred species, within the scope of the claims appended hereto, which reside within the range of about 90% to 100% homology to SEQ ID NO: 1 as well as the binding site thereof. As noted above, N- or C-terminal extensions shall not be construed as affecting homology. Thus, when comparing two sequences, the reference sequence is generally the shorter of the two sequences. This means that, for example, if a sequence of 50 nucleotides in length with precise identity to a 50 nucleotide region within a 100 nucleotide polynucleotide is compared, there is 100% homology as opposed to only 50% homology.

Although the natural polypeptide of SEQ ID NO: 1 and a variant polypeptide may only possess a certain percentage identity, e.g., 90%, they are actually likely to possess a higher degree of similarity, depending on the number of dissimilar codons that are conservative changes. Conservative amino acid substitutions can frequently be made in a protein without altering either the conformation or function of the protein. Similarity between two sequences includes direct matches as well a conserved amino acid substitutes which possess similar structural or chemical properties, e.g., similar charge as described in Table 4.

Percentage similarity (conservative substitutions) between two polypeptides may also be scored by comparing the amino acid sequences of the two polypeptides by using programs well known in the art, including the BESTFIT program, by employing default settings for determining similarity.

A further embodiment of the invention is a crystal comprising the coordinates of FIG. 4, wherein the amino acid sequence is represented by SEQ ID NO:1 or 2. A further embodiment of the invention is a crystal comprising the coordinates of FIG. 4, wherein the amino acid sequence is at least 75%, 80%, 85%, 90%, 95%, or 98% homologous to the amino acid sequence represented by SEQ ID NO:1 or 2.

Various methods for obtaining atomic coordinates of structurally homologous molecules and molecular complexes using homology modeling are disclosed in U.S. Pat. No. 6,356,845, which is hereby incorporated by reference in its entirety.

VII. Structure Based Drug Design

Once the three-dimensional structure of a crystal comprising a PDE1B protein, a functional domain thereof, homologue or variant thereof, is determined, a potential ligand (antagonist or agonist) may be examined through the use of computer modeling using a docking program such as GRAM, DOCK, or AUTODOCK (see, for example, Morris et al., J. Computational Chemistry, 19:1639-62 (1998); as well as the website http://www.accelrys.com/insight). This procedure can include in silico fitting of potential ligands to the PDE1B crystal structure to ascertain how well the shape and the chemical structure of the potential ligand will complement or interfere with the catalytic domain of PDE1B (Bugg et al., Scientific American, December:92-98 (1993); West et al., TIPS, 16:67-74 (1995)). Computer programs can also be employed to estimate the attraction, repulsion, and steric hindrance of the ligand to the binding site. Generally the tighter the fit (e.g., the lower the steric hindrance, and/or the greater the attractive force) the more potent the potential drug will be since these properties are consistent with a tighter binding constant. Furthermore, the more specificity in the design of a potential drug the more likely that the drug will not interfere with the properties of other proteins. This will minimize potential side-effects due to unwanted interactions with other proteins.

One embodiment of the present invention relates to a method of identifying an agent that binds to a binding site on PDE1B catalytic domain wherein the binding site comprises amino acid residues His223, His373, Thr385, Leu388, Ser420, Gln421, and Phe424 of SEQ ID NO:1 comprising contacting PDE1B with a test ligand under conditions suitable for binding of the test ligand to the binding site, and determining whether the test ligand binds in the binding site, wherein if binding occurs, the test ligand is an agent that binds in the binding site. In certain embodiments, the testing may be carried out in silico using a variety of molecular modeling software algorithms including, but not limited to, DOCK, ALADDIN, CHARMM simulations, AFFINITY, C2-LIGAND FIT, Catalyst, LUDI, CAVEAT, and CONCORD. (Brooks, et al. CHARMM: a program for macromolecular energy, minimization, and dynamics calculations. J. Comp. Chem., 1983, 4:187-217; E. C. Meng, B. K. Shoichet & I. D. Kuntz. Automated docking with grid-based energy evaluation. J. Comp. Chem., 1992, 13:505-524).

In another embodiment, a potential ligand may be obtained by screening a random peptide library produced by a recombinant bacteriophage (Scott and Smith, Science, 249:386-390 (1990); Cwirla et al., Proc. Natl. Acad. Sci., 87:6378-82 (1990); Devlin et al., Science, 249:404-06 (1990)), or a chemical library, or the like. A ligand selected in this manner can be then be systematically modified by computer modeling programs until one or more promising potential ligands are identified. Such analysis has been shown to be effective in the development of HIV protease inhibitors (Lam et al., Science 263:380-84 (1994); Wlodawer et al., Ann. Rev. Biochem. 62:543-85 (1993); Appelt, Perspectives in Drug Discovery and Design 1:23-48 (1993); Erickson, Perspectives in Drug Discovery and Design 1:109-28 (1993)).

Such computer modeling allows the selection of a finite number of rational chemical modifications, as opposed to the countless number of essentially random chemical modifications that could be made, and of which any one might lead to a useful drug. Each chemical modification requires additional chemical steps, which while being reasonable for the synthesis of a finite number of compounds, quickly becomes overwhelming if all possible modifications needed to be synthesized are actually synthesized. Thus, through the use of the three-dimensional structure disclosed herein and computer modeling, a large number of these compounds can be rapidly screened on a computer monitor screen, and a few likely candidates can be determined without the laborious synthesis of untold numbers of compounds.

Once a potential ligand (agonist or antagonist) is identified, it can be either selected from a library of chemicals, or alternatively, the potential ligand may be synthesized de novo. As mentioned above, the de novo synthesis of one or even a relatively small group of specific compounds is reasonable in the art of drug design. The prospective drug can be placed into any standard binding assay described below to test its effect on PDE1B interaction.

When a suitable drug is identified, a supplemental crystal can be grown which comprises a protein-ligand complex formed between a PDE1B protein and the drug. Preferably the crystal effectively diffracts X-rays allowing the determination of the atomic coordinates of the protein-ligand complex to a resolution of less than 5.0 Angstroms, more preferably less than 3.0 Angstroms, and even more preferably less than 2.0 Angstroms. The three-dimensional structure of the supplemental crystal can be determined by Molecular Replacement Analysis. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: X-PLOR and AMORE (Navaza, Acta Crystallographics ASO, 157-63 (1994)). Once the position and orientation are known, an electron density map can be calculated using the search model to provide X-ray phases. Thereafter, the electron density is inspected for structural differences, and the search model is modified to conform to the new structure. Using this approach, it is possible to use the claimed structure of PDE1B to solve the three-dimensional structures of any such PDE1B complexed with a new ligand. Other computer programs that can be used to solve the structures of such STAT crystals include QUANTA, CHARMM; INSIGHT; SYBYL; MACROMODEL; and ICM.

For all of the drug screening assays described herein further refinements to the structure of the drug will generally be necessary and can be made by the successive iterations of any and/or all of the steps provided by the particular drug screening assay.

Various in silico methods for screening, designing or selecting ligands are disclosed in U.S. Pat. No. 6,356,845, the pertinent disclosure of which is incorporated by reference herein.

VIII. Ligands

In one aspect, the present invention discloses ligands which interact with a binding site of the PDE1B catalytic domain defined by a set of points having a root mean square deviation of less than about 2.0 Å from points representing the backbone atoms of the amino acids represented by the structure coordinates listed in FIG. 4. A further embodiment of the present invention comprises binding agents which interact with a binding site of PDE1B defined by a set of points having a root mean square deviation of less than about 2.0, 1.7, 1.5, 1.2, 1.0, 0.7, 0.5, or even 0.2 Å from points representing the backbone atoms of the amino acids represented by the structure coordinates listed in FIG. 4. Such embodiments represent variants of the PDE1B crystal.

In another aspect, the present invention describes ligands which bind to a correctly folded polypeptides comprising an amino acid sequence spanning amino acids 142 to 507 listed in SEQ ID NO:1, or a homologue, or a variant thereof. In certain embodiments, the ligand is a competitive or uncompetitive inhibitor of PDE1B. In certain embodiments the ligand inhibits PDE1B with an IC₅₀ or Ki of less than about 10 μM, 1 μM, 500 nM, 100 nM, 50 nM or 10 nM. In certain embodiments, the ligand inhibits PDE1B with a Kᵢ that is less than about one-half, one-fifth, or one-tenth the Kᵢ that the substance has for inhibition of any other PDE enzyme. In other words, the substance inhibits PDE1B activity to the same degree at a concentration of about one-half, one-fifth, one-tenth or less than the concentration required for any other PDE enzyme.

One embodiment of the present invention relates to ligands, such as proteins, peptides, peptidomimetics, small organic molecules, etc., designed or developed with reference to the crystal structure of PDE1B as represented by the coordinates presented herein in FIG. 4, and portions thereof. Such binding agents interact with the binding site of the PDE1B represented by one or more amino acid residues selected from His223, His373, Thr385, Leu388, Ser420, Gln421, and Phe424.

IX. Machine Readable Storage Media

Transformation of the structure coordinates for all or a portion of PDE1B, or the PDE1B/ligand complex or one of its binding pockets, for structurally homologous molecules as defined below, or for the structural equivalents of any of these molecules or molecular complexes as defined above, into three-dimensional graphical representations of the molecule or complex can be conveniently achieved through the use of commercially-available software.

The invention thus further provides a machine-readable storage medium comprising a data storage material encoded with machine readable data which, when using a machine programmed with instructions for using said data, is capable of displaying a graphical three-dimensional representation of any of the molecule or molecular complexes of this invention that have been described above. In a preferred embodiment, the machine-readable data storage medium comprises a data storage material encoded with machine readable data which, when using a machine programmed with instructions for using said data, is capable of displaying a graphical three-dimensional representation of a molecule or molecular complex comprising all or any parts of a PDE1B C-terminal catalytic domain or binding pocket, as defined above. In another preferred embodiment, the machine-readable data storage medium is capable of displaying a graphical three-dimensional representation of a molecule or molecular complex defined by the structure coordinates of the amino acids listed in FIG. 4, plus or minus a root mean square deviation from the backbone atoms of said amino acids of not more than 2.0 Å.

In an alternative embodiment, the machine-readable data storage medium comprises a data storage material encoded with a first set of machine readable data which comprises the Fourier transform of the structural coordinates set forth in FIG. 4, and which, when using a machine programmed with instructions for using said data, can be combined with a second set of machine readable data comprising the X-ray diffraction pattern of a molecule or molecular complex to determine at least a portion of the structural coordinates corresponding to the second set of machine readable data.

For example, a system for reading a data storage medium may include a computer comprising a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may comprise CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding pocket of this invention using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

### EXAMPLES

The present invention is further illustrated by the following examples which should not be construed as limiting in any way. The contents of all cited references (including literature references, issued patents, published patent applications as cited throughout this application) are hereby expressly incorporated by reference in their entirety.

The practice of the present invention will employ, unless otherwise indicated, conventional techniques of cell biology, cell culture, molecular biology, microbiology and recombinant DNA, X-ray crystallography, and molecular modeling which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Crystallography Made Crystal Clear: A Guide for Users of Macromolecular Models (Gale Rhodes, 2ND Ed. San Diego; Academic Press, 2000).

### Example 1

### Construction and Expression of PDE1B Wild Type Catalytic Domain

A construct of human PDE1B was generated by PCR and subcloned into pFastBac-1 in order to generate recombinant baculovirus using the Bac-to-Bac system (Gibco). The final protein encompasses the catalytic region starting at T142 and extending to Q507. The protein was expressed in SF21 insect cells infected with the recombinant baculovirus at a MOI of 0.1 and harvested 72 hrs. post infection. Pellets of infected cells were frozen at −80° C. for transfer to purification.

### Example 2

### Purification of PDE1B Wild Type Catalytic Domain

Insect cell paste was resuspended (3 ml/g) in ice-cold lysis buffer (25 mM HEPES, pH 7.5; 5 mM Tris (2-carboxytheyl) phosphine hydrochloride (TCEP; Fluka); EDTA-free protease inhibitors (Roche Biochemicals; as per manufacturer's recommendations); 1 μg/ml leupeptin (Sigma); 1 mM PMSF (Roche Biochemicals); 10 μM E-64 (Roche Biochemicals) and lysed by one passage through a chilled microfluidizer (M110L, Microfluidics International Corp., Newton, Mass.) at a chamber pressure of 18 kpsi. The lysate was centrifuged at 43,000×g for 30 min at 4° C. The supernatant was concentrated two-fold using an ultrafiltration apparatus (10K MWCO hollow fiber filter; AG Technology Corp., Needham, Mass.) and subsequently diafiltered with 5 volumes lysis buffer. The diafiltrate was loaded onto an SP Sepharose (Amersham Biosciences) column (Amersham Biosciences XK50/20; 50 mm i.d. ×20 cm) in tandem with a Q XL Sepharose (Amersham Biosciences) column (Amersham Biosciences XK50/20; 50 mm i.d. ×20 cm) pre-equilibrated with lysis buffer. After loading was complete and the columns were washed to baseline, the SP Sepharose was disconnected from the series and protein was eluted from the QXL resin over 20 column volumes at 1 ml/min with a 0-1 M NaCl linear gradient. Fractions containing PDE activity (Johnson et al., Analytical Biochemistry, 162:291-95 (1987)) were pooled and bound to a Blue Sepharose 6 Fast Flow (Amersham Biosciences) column (Amersham Biosciences XK26/20; 26 mm i.d. ×20 cm) pre-equilibrated with lysis buffer. Material enriched for PDE 1B was eluted with lysis buffer containing 20 mM cGMP. The Blue Sepharose purified material was further purified by a MonoQ HR 1010 column (Amersham Biosciences) equilibrated in lysis buffer and eluted over 20 column volumes with a 0-1M NaCl gradient. PDE 1B elutes in two peaks that are kept separate. Peak 2 fractions are pooled and concentrated to 5 ml and loaded onto a Superdex HiLoad 1660 column (Amersham Biosciences) equilibrated in 25 mM HEPES, pH 7.5; 5 mM TCEP; 10 μM E64; 1 ug/ml leupeptin, and 350 mM NaCl. Fractions are pooled on the basis of purity analyzed by Coomassie-stained SDS-PAGE.

### Example 3

### Crystallization of PDE1B Wild-Type Catalytic Domain with Compound 109

Crystals of PDE-1B-13 Complexed with compound 109 were grown with vapor diffusion. Large crystal (0.2×0.3×0.4 mm) appeared after 1-3 days when the protein (10 mg/ml PDE-1B with 250 μM compound 109) was mixed with an equal volume of reservoir (0.1 M Tris-HCl, pH 8.5, 0.2 M MgCl₂, 15% PEG 8000) at 22° C.

### Example 4

### X-Ray Data Collection, Structure Determination and Refinement of PDE1B:Compound 109 Complex

Crystals were transferred to a cryoprotectant solution, made up of the reservoir solution, with 15% glycerol, and then flash-frozen in a stream of cold nitrogen gas at 100K. A full data set was collected from one crystal frozen in this manner on a Rigaku RAXIS IIc detector, mounted on a Rigaku RU-200 generator with Osmic optics. Data were processed using the HKL suite of software (Otwinowski & Minor, Methods Enzymol. 276(Macromolecular Crystallography, Part A): 307-26 (1997)). Data collection statistics are summarized in Table 5a.

TABLE 5a
Data statistics

| Resolution range | | 20.0-2.1 Å |
|---|---|---|
| Number of observations | Total | 219,753 |
| | Unique | 30,621 |
| Completeness (%) | | 98.0 (97.4)¹ |
| I/σ (I) | | 17.3 (3.3)¹ |
| R_{sym} | | 0.042 (0.52)^{1,2} |

¹Numbers in parentheses refer to the highest resolution range (2.10-2.17 Å)
²R_{sym} = Σ (I − <I>)/Σ<I>

The crystals belong to space group P4₃2₁2 with unit cell dimensions a=87.47, b=87.47, c=135.03 Å, α=β=γ→90.0°. They contain 1 molecule of the polypeptide, and one molecule of the inhibitor per asymmetric unit.

The structure was solved by the method of molecular replacement, using the program EPMR (Kissinger et al., Acta Crystallographica, D55:484-91 (1999)). The search model consisted of only the backbone atoms of PDE4B taken from PDB entry 1FOJ (Xu et al., supra), residues 152 to 461. A clear solution to the rotation and translation function searches was found using diffraction data limited to 4 Å resolution.

A homology model of PDE1B was then positioned according to the top rotation/translation search, and subjected to refinement, and a combination of automatic and manual refitting. Automatic refitting was carried out using the program ArpWarp (http://www.arp-warp.org) in combination with Refmac (Murshudov et al., Acta Cryst. D53:240-55 (1997)), and manual fitting used the program 0 Refinement in Refmac was carried out using all data in the resolution range 20.0-2.1 Å. Partial structure factors from a bulk-solvent model and anisotropic B-factor correction were supplied throughout the refinement. The R-factor for the current model is 0.21 (free R-factor, 7% of the data, 0.23). The refinement statistics are summarized in Table 5b.

TABLE 5b
Refinement statistics

| | |
|---|---|
| Nr. of reflections used (%) | 28,188 (97.7%) |
| Nr. of reflections used for R_{free} | 2,359 (7.7%) |
| R_{cryst}/R_{free} | 0.213/0.235³ |
| Number of atoms | 2,724 |

³R = Σ∥F_{obs}| − k|F_{calc}∥/Σ|F_{obs}|

The current model contains 321 out of 366 amino acid residues calculated on the basis of the construct. No interpretable electron density is observed for residues 142-147, 445-478, and 503-507 . In addition, the model contains one Zn²⁺ ion, one Mg²⁺ ion, one molecule of the inhibitor compound 109, and 101 water molecules.

### Equivalents

While specific embodiments of the subject invention have been discussed, the above specification is illustrative and not restrictive. Many variations of the invention will become apparent to those skilled in the art upon review of this specification. The appended claims should be interpreted by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

1. A phosphodiesterase 1B (PDE1B) crystal.

2. The PDE1B crystal of claim 1 which is derived from a mammal.

3. The PDE1B crystal of claims 2 comprising SEQ ID NO:1, or a homologue or variant thereof.

4. A crystal of a PDE1B/PDE1B ligand complex.

5. The crystal complex of claim 4 wherein said ligand is an antagonist or an inhibitor.

6. A crystal complex comprising a polypeptide with an amino acid sequence spanning amino acids Thr142 to Gln507 listed in SEQ ID NO:1, or a homologue or variant thereof.

7. The crystal complex of claim 6, wherein the homologue or variant has an amino acid identity of at least 98%, 95%, 90%, 85%, 80%, or 75% with a polypeptide having an amino acid sequence spanning amino acids Thr142 to Gln507 listed in SEQ ID NO:1.

8. The crystal complex of claim 7, wherein said crystal further comprises compound 109 and said crystal has the atomic coordinates listed in FIG. 4.

9. The crystal complex of claim 6, wherein the homologue or variant thereof has a protein backbone comprising the atomic coordinates, or portions thereof, that are within a root mean square of ±2.0, 1.7, 1.5, 1.2, 1.0, 0.7, 0.5, or even 0.2 Å of the atomic coordinates, or portions thereof, listed in FIG. 4.

10. A polypeptide comprising the amino acid sequence set forth in SEQ ID NO: 1 or a homologue or variant thereof, wherein the molecules are arranged in a crystalline manner belonging to space group P4₃2₁2 with unit cell dimensions a=87.47 Å, b=87.47 Å, c=135.03 Å, α=β=γ=90.0°, and which effectively diffracts X-rays for determination of the atomic coordinates of PDE1B polypeptide to a resolution of about 1.8 Å.

11. A polypeptide consisting essentially of the catalytic domain of PDE1b.

12. A computer for producing a three-dimensional representation of a polypeptide with an amino acid sequence spanning amino acids Thr142 to Gln507 listed in SEQ ID NO:1, or a homologue, or a variant thereof comprising:
a computer-readable data storage medium comprising a data storage material encoded with computer-readable data, wherein said data comprises the structure coordinates of FIG. 4, or portions thereof;
a working memory for storing instructions for processing said computer-readable data;
a central-processing unit coupled to said working memory and to said computer-readable data storage medium for processing said computer-machine readable data into said three-dimensional representation; and
a display coupled to said central-processing unit for displaying said representation.

13. A computer for producing a three-dimensional representation of a molecule or molecular complex comprising the atomic coordinates in FIG. 4 comprising:
a computer-readable data storage medium comprising a data storage material encoded with computer-readable data, wherein said data comprises the structure coordinates of FIG. 4, or portions thereof;
a working memory for storing instructions for processing said computer-readable data;
a central-processing unit coupled to said working memory and to said computer-readable data storage medium for processing said computer-machine readable data into said three-dimensional representation; and
a display coupled to said central-processing unit for displaying said representation.

14. A computer for producing a three-dimensional representation of a molecule or molecular complex comprising the atomic coordinates having a root mean square deviation of less than 2.0, 1.7, 1.5, 1.2, 1.0, 0.7, 0.5, or 0.2 Å from the atomic coordinates for the carbon backbone atoms listed in FIG. 4 comprising:
a computer-readable data storage medium comprising a data storage material encoded with computer-readable data, wherein said data comprises the structure coordinates of FIG. 4, or portions thereof;
a working memory for storing instructions for processing said computer-readable data;
a central-processing unit coupled to said working memory and to said computer-readable data storage medium for processing said computer-machine readable data into said three-dimensional representation; and
a display coupled to said central-processing unit for displaying said representation.

15. A computer for producing a three-dimensional representation of a molecule or molecular complex comprising a binding site defined by the structure coordinates in FIG. 4, or a the structural coordinates of a portion of the residues in FIG. 4, or the structural coordinates of one or more PDE1B amino acids in SEQ ID NO:1 selected from His223, His373, Thr385, Leu388, Ser420, Gln421, and Phe424, wherein said computer comprises:
a computer-readable data storage medium comprising a data storage material encoded with computer-readable data, wherein said data comprises the structure coordinates of FIG. 4, or portions thereof;
a working memory for storing instructions for processing said computer-readable data;
a central-processing unit coupled to said working memory and to said computer-readable data storage medium for processing said computer-machine readable data into said three-dimensional representation; and
a display coupled to said central-processing unit for displaying said representation.

16. The PDE1B crystal of claim 1 having the atomic coordinates set out in FIG. 4, or a derivative expressed in any reference frame.

17. A method for generating the 3-D atomic coordinates of protein homologues of PDE1B using the X-ray coordinates of PDE1B described in FIG. 4, said method comprising:
identifying the sequences of one or more proteins which are homologues of PDE1B;
aligning the homologue sequences with the sequence of PDE1B (SEQ ID NO:1);
identifying structurally conserved and structurally variable regions between the homologue sequences, and PDE1B (SEQ ID NO: 1);
generating 3-D coordinates for structurally conserved residues, variable regions and side-chains of the homologue sequences from those of PDE1B; and
combining the 3-D coordinates of the conserved residues, variable regions and side-chain conformations to generate a full or partial 3-D coordinates for said homologue sequences.

18. A method for identifying a potential ligands for PDELB, or homologues, analogues or variants thereof, comprising the steps of:
displaying three dimensional structure of PDE1B enzyme, or portions thereof, as defined by atomic coordinates in FIG. 4, on a computer display screen;
optionally replacing one or more PDE1B enzyme amino acid residues listed in SEQ ID NO:1, or one or more of the amino acids listed in Tables 1-3, or one or more amino acid residues selected from His223, His373, Thr385, Leu388, Ser420, Gln421, and Phe424, in said three-dimensional structure with a different naturally occurring amino acid or an unnatural amino acid;
employing said three-dimensional structure to design or select said ligand;
contacting said ligand with PDE1B, or variant thereof, in the presence of one or more substrates; and
measuring the ability of said ligand to modulate the activity PDE1B.

19. The method of claim 18 further comprising the steps of:
computationally modifying the structure of the ligand; and
computationally determining the fit of the modified ligand with the three-dimensional coordinates of PDE1B set forth in FIG. 4, or portions thereof.

20. A method for treating psychological disorders comprising administering to a patient in need of treatment the pharmaceutical compositions of ligands identified by structure-based drug design using the atomic coordinates substantially similar to, or portions of, the coordinated listed in FIG. 4.

21. The method of claim 20, wherein the psychological disorders are selected from multiple variants of schizophrenia, anxiety disorders, movement disorders selected from Huntington's disease, Parkinson's disease and dyskinesia, alcohol and drug addictions, cognitive deficiencies, and mood disorders.

22. An expression vector useful in a method for preparing a purified catalytic domain of PDE1B comprising a polypeptide with an amino acid sequence spanning amino acids Thr142 to Gln507 listed in SEQ ID NO:1, or a homologue or variant thereof.


SEQUENCE LISTING (2 sequences)

SEQ ID NO: 1 - protein, 536 aa, Homo sapiens
MELSPRSPPEMLEESDCPSPLELKSAPSKKMWIKLRSLLRYMVKQLENGEINIEELKKNLEYTASLLEAVYIDETRQILDTEDELQELRSDAVPSEVRDWLASTFTQQARAKGRRAEEKPKFRSIVHAVQAGIFVERMFRRTYTSVGPTYSTAVLNCLKNLDLWCFDVFSLNQAADDHALRTIVFELLTRHNLISRFKIPTVFLMSFLDALETGYGKYKNPYHNQIHAADVTQTVHCFLLRTGMVHCLSEIELLAIIFAAAIHDYEHTGTTNSFHIQTKSECAIVYNDRSVLENHHISSVFRLMQDDEMNIFINLTKDEFVELRALVIEMVLATDMSCHFQQVKTMKTALQQLERIDKPKALSLLLHAADISHPTKQWLVHSRWTKALMEEFFRQGDKEAELGLPFSPLCDRTSTLVAQSQIGFIDFIVEPTFSVLTDVAEKSVQPLADEDSKSKNQPSFQWRQPSLDVEVGDPNPDVVSFRSTWVKRIQENKQKWKERAASGITNQMSIDELSPCEEEAPPSPAEDEHNQNGNLD

SEQ ID NO: 2 - protein, 366 aa, Homo sapiens
TYTSVGPTYSTAVLNCLKNLDLWCFDVFSLNQAADDHALRTIVFELLTRHNLISRFKIPTVFLMSFLDALETGYGKYKNPYHNQIHAADVTQTVHCFLLRTGMVHCLSEIELLAIIFAAAIHDYEHTGTTNSFHIQTKSECAIVYNDRSVLENHHISSVFRLMQDDEMNIFINLTKDEFVELRALVIEMVLATDMSCHFQQVKTMKTALQQLERIDKPKALSLLLHAADISHPTKQWLVHSRWTKALMEEFFRQGDKEAELGLPFSPLCDRTSTLVAQSQIGFIDFIVEPTFSVLTDVAEKSVQPLADEDSKSKNQPSFQWRQPSLDVEVGDPNPDVVSFRSTWVKRIQENKQKWKERAASGITNQ